(19) United States
(12) Patent Application Publication
(10) Pub. No.: US 2022/0347216 A1
(43) Pub. Date: Nov. 3, 2022

(54) NK CELL IMMUNOTHERAPY COMPOSITIONS, METHODS OF MAKING AND METHODS OF USING SAME

(71) Applicant: OHIO STATE INNOVATION FOUNDATION, Columbus, OH (US)

(72) Inventors: Sumithira VASU, Columbus, OH (US); Lynn O'DONNELL, Columbus, OH (US); Hillary BRADBURY, Columbus, OH (US)

(21) Appl. No.: 17/642,622

(22) PCT Filed: Sep. 14, 2020
(86) PCT No.: PCT/US20/50742

§ 371 Date: Mar. 11, 2022

Related U.S. Application Data
(60) Provisional application No. 62/900,245, filed on Sep. 13, 2019.

(51) Int. Cl. A61K 35/17; A61K 47/20; A61K 47/42; C12N 5/0783

(52) U.S. Cl. CPC A61K 35/17; A61K 47/20; A61K 47/42; C12N 5/0646; C12N 2501/2302

(57) ABSTRACT

Natural Killer (NK) cells represent a potent therapeutic for patients suffering from cancer or infectious diseases. NK cells typically represent a minor fraction of the lymphocytes and express multiple receptors that interact with human leukocyte antigen (HLA). Disclosed are methods of expanding NK cells and compositions of NK cells for administration in patients. The methods described herein can be used to identify donor NK cells for administration to a recipient subject. The NK cell compositions disclosed herein can be used to treat a number of diseases including cancer and infectious diseases.

### CROSS REFERENCE TO RELATED APPLICATIONS

This application claims the benefit of U.S. Provisional Application 62/900,245 filed on Sep. 13, 2019, of which is hereby incorporated by reference in its entirety.

### TECHNICAL FIELD

The present disclosure generally relates to the field of immunotherapy, particularly adoptive Natural Killer (NK) cell immunotherapy.

### BACKGROUND

Natural Killer (NK) cells are a key component of anti-tumor immunity that, unlike T cells, recognize cancer in a manner that ignores self, is not antigen-specific nor MHC-restricted, and induces a pro-inflammatory environment that primes adaptive immunity. NK cell number and function are low in a wide variety of malignancies, traffic poorly to tumor sites or across the blood-brain barrier, and are further depleted by chemotherapy, radiation, and surgical anesthesia. This provides a strong rational for restoring NK cell function through adoptive transfer.

NK cells typically represent only a minor fraction of human lymphocytes or white blood cells (WBCs). Standard procedures used to collect blood or plasma from donors typically yields low numbers of isolated NK cells. Thus, a major limitation to the development of NK cell therapies has been the inability to generate adequate numbers of NK cells.

Human NK cells express multiple receptors that interact with human leukocyte antigen (HLA) class I molecules. These receptors belong to one of two major protein superfamilies, the immunoglobulin superfamily or the C type lectin superfamily. The ability of NK cells to discriminate normal from pathologic self-tissues is largely explained by the inhibitory function of the killer cell immunoglobulin-like receptor (KIR) family which predominantly recognize classical HLA class I molecules on potential targets. This self-major histocompatibility complex (MHC) recognition confers functional competence on the NK cell to be triggered through their activation receptors, a process termed licensing. As a result, licensed NK cells with self-MHC-specific receptors are more readily activated as compared with unlicensed NK cells without self-MHC-specific receptors. Different KIR family members interact with discrete HLA class I allotypes and have extensive genetic diversity. Similarly, NK cells simultaneously express multiple different receptors with different specificities. As a result, any attempt to utilize NK cells in an adoptive immunotherapy has to contend with the compatibility between the NK cell donor and recipient. Thus, there is a need for novel methods for expanding NK cells to generate adequate cell numbers for clinical efficacy. There is also a need for novel NK cell therapeutic compositions and methods of screening and dosing recipients suffering from cancer or disease.

### SUMMARY

The present disclosure is, in part, related to novel, methods and compositions of universal donor NK cells that can be used for therapeutic administration to a recipient subject in need thereof.

As described herein, in some embodiments are methods of selecting universal donor NK cells for therapeutic administration to a recipient subject in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4 and (c) selecting the candidate NK cells as a universal donor NK cell for the therapeutic administration when (i) the HLA genotype indicates the presence of at least two HLA alleles HLA C1, C2, and Bw4 and/or (ii) the KIR genotype indicates the presence of at least three activating KIRs 2DS1/2, 2DS3/5, 3DS1, and/or 2DS4.

Also disclosed herein, in some embodiments are methods of screening a population of candidate NK cells from a donor to identify universal NK donor cells in the population for providing a source of NK cells for therapeutic administration to recipient subjects in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2 or 2DL3, and/or 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; wherein candidate NK cells comprising (i) at least two HLA alleles HLA C1, C2, and Bw4 and therefore comprising at least one of the variably inherited inhibitory KIRs 2DL1, 2DL2 or 2DL3, and/or 3DL1 and/or (ii) at least three activating KIRs 2DS1/2, 2DS3/5, 3DS1, and/or 2DS4 are universal NK cells.

According to one non-limiting aspect of the present disclosure, the method of screening a population of NK cells or a method of selecting universal donor NK cells for therapeutic administration to a recipient subject of any preceding aspect, wherein the selected universal donor NK cells are histologically optimized for at least 50%-85% of recipient subjects.

According to another non-limiting aspect of the present disclosure, the methods of screening a population of NK cells or methods of selecting universal donor NK cells for therapeutic administration to a recipient subject of any preceding aspect, further comprising obtaining or having obtained the Cytomegalovirus (CMV) seropositivity of the candidate NK cells; and wherein the NK candidate NK cells are further selected when the NK cell donor is seropositive for CMV or the NK cells from the NK cell donor have high NKG2C expression compared to a reference level of NKG2C expression.

Also disclosed herein, the isolated universal donor NK cells are selected or screened by the method of any preceding embodiment or aspect.

In one embodiment, the NK cells of any preceding embodiment, wherein the isolated NK cells are NKG2C+.

Also disclosed herein are isolated universal NK cells of any preceding aspect, wherein the NK cell is activated by incubating the universal donor NK cells in vitro in the presence of interleukin (IL)-21. The IL-21 can be soluble, presented in solution, or present as membrane bound agent on the surface of plasma membrane (PM) particles, exosome (EX), or feeder cells (FC). In one embodiment, the IL-21 used in the in vitro activation comprises soluble IL-21, IL-21-expressing FCs, IL-21 PM particles, and/or IL-21 EXs.

According to another non-limiting aspect of the present disclosure, are methods of treating, preventing, inhibiting, and/or reducing a cancer, metastasis, or an infectious disease in a subject comprising administering to the recipient subject a donor NK cell selected by or screened by the method of any preceding aspect; or administering to the recipient subject the isolated universal NK cell of any preceding aspect. For example, in one aspect, disclosed herein are methods of treating a cancer or an infectious disease in a subject comprising (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1; (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; and (c) selecting the candidate NK cells as a universal donor NK cell for the therapeutic administration when (i) the HLA genotype indicates the presence of at least two HLA alleles HLA C1, C2, and Bw4; and (ii) the KIR genotype indicates the presence of at least three activating KIRs 2DS1/2, 2DS3/5, 3DS1, and/or 2DS4.

Also disclosed herein are methods treating a cancer or an infectious disease of any preceding aspect, wherein the selected universal donor NK cells are histologically optimized for at least 50%-85% of recipient subjects.

Also disclosed herein are methods treating a cancer or an infectious disease of any preceding aspect, further comprising obtaining or having obtained the CMV seropositivity of the candidate NK cells; and wherein the NK candidate NK cells are further selected when the NK cell donor is seropositive for CMV or the NK cells from the NK cell donor have high NKG2C expression compared to a reference level of NKG2C expression.

According to another non-limiting aspect of the present disclosure, are methods treating a cancer or an infectious disease of any preceding aspect, further comprising incubating the selected universal donor NK cells in vitro in the presence of IL-21. In one aspect, the IL-21 used in the in vitro culture comprises soluble IL-21, FCs expressing membrane-bound IL-21, PMs, and/or EXs.

Also disclosed herein are methods for preparing a population of universal donor NK cells for therapeutic administration to a recipient subject in need thereof, the method comprising: (a) obtaining an initial population of NK cells from a NK cell donor, wherein the NK cell donor has a genotype indicating the presence of (i) at least two of variably inherited activating KIRs 2DS1/2, 2DS3/5, 3DS1, and/or 2DS4; and (ii) at least one, two, or all three HLA alleles comprising of C1, C2, and Bw4 alleles; and (b) exposing the initial population of NK cells to IL-21 in vitro for a time and under conditions sufficient to expand the initial population of NK cells.

According to another non-limiting aspect of the present disclosure, are populations of the NK cells of any preceding aspect, wherein the isolated NK cells are NKG2C+ or CMV seropositive.

The method of preparing a population of NK cells, wherein exposing the initial population of NK cells to IL-21 comprises contacting the NK cells in vitro with at least one of soluble IL-21, FC21s (also referred to herein as FCs), PM21s, and/or EX21s. For example, disclosed herein are methods of preparing a population of NK cells, wherein the IL-21 present on FC21s, PM21s, and EX21s comprises a form of IL-21 selected from (a) an engineered membrane bound form for IL-21, (b) IL-21 chemically conjugated to the surface of FC21s, PM21s, or EX21s, or (c) or IL-21 in solution mixed to be in co-contact with the NK cells. In one aspect, any one of the FC21, PM21 or EX21 further comprise (a) an NK stimulatory ligand selected from IL-2, IL-12, IL-18, IL-15, IL-7, ULBP, MICA, OX40L, NKG2D agonists, Delta-1, Notch ligands, NKp46 agonists, NKp44 agonists, NKp30 agonists, other NCR agonists, CD16 agonists; or (b) membrane bound transforming growth factor beta (TGFβ).

In one aspect, disclosed herein is a population of universal donor NK cells prepared by the method of any preceding aspect. In one aspect, the population of NK cells is characterized by increased ability to produce and secrete anti-tumor cytokines of interferon gamma (IFNγ) or tumor necrosis factor alpha (TNFα).

In another aspect, the expanded population of NK cells is characterized by one or more of the following: increased expression of NKG2D, increased expression of CD16, increased expression of NKp46, and/or increased KIR expression.

Another embodiment disclosed herein are engineered NK cells or cell lines, wherein the NK cells have been transformed to express one, two, or more HLA alleles comprising C1, C2 or Bw4 (for example an NK cell or cell line that expresses C1, C2, and Bw4) and/or transformed to express of one, two, three, four, five or more variably inherited activating KIRs comprising 2DS1/2, 2DS3/5, 3DS1, or 2DS4.

In one embodiment, is a Final NK cell composition for use in the treatment of a cancer in a subject in need thereof.

In another embodiment, disclosed herein is a Final NK cell composition for use in the treatment of a disease in a subject in need thereof.

In one aspect, disclosed herein is a method of modifying T cell-depleted mononuclear cells, the method comprising freezing the T cell-depleted mononuclear cells in a cryoprotectant media, wherein the cryoprotectant media comprises 10% dimethyl sulfoxide (DMSO), 40% fetal bovine serum (FBS), and 50% RPMI Complete Media.

Also disclosed herein is a composition comprising T cell-depleted mononuclear cells in a cryoprotectant media, wherein the cryoprotectant media comprises 10% dimethyl sulfoxide (DMSO), 40% fetal bovine serum (FBS), and 50% RPMI Complete Media.

In one embodiment, is a method of expanding natural killer (NK) cells, the method comprising: growing a culture of T cell-depleted mononuclear cells, the culture comprising a cell nutrient media and at least one activating agent; splitting the culture of T cell-depleted mononuclear cells at least once after seven days; replacing the cell nutrient media at least once after seven days; and harvesting Final NK cells after about 14 days, the Final NK cells comprising less than about 5% of CD3+ cells/white blood cells (WBCs) and more than about 90% of CD3-CD56+ cells/WBCs.

In another embodiment, the Final NK cells comprises an average of 2,800 times more NK cells compared to the T cell-depleted mononuclear cells.

In one aspect, the at least one activating agent is provided to the culture of T cell-depleted mononuclear cells by a delivery vehicle.

In another aspect, the Final NK cells comprise less than about 5% of CD3+ cells/WBCs and more than about 95% of CD3-CD56+ cells/WBCs.

Also disclosed herein is a therapeutic composition comprising: cryopreserved Final NK cells including less than about 5% of CD3+ cells/WBCs and more than about 90% of CD3-CD56+ cells/WBCs, wherein the cryopreserved Final NK cells are a known HLA-type; and wherein the composition of Final NK cells comprises more than one concentration of cryopreserved Final NK cells.

In one embodiment, the Final NK cells are selected from A cells/mL, B cells/mL, or C cells/mL.

In another embodiment, disclosed herein, the composition comprises less than about 1% of CD3+ cells/WBCs and more than about 95% of CD3-CD56+ cells/WBCs.

Also disclosed, is a method of storing a therapeutic composition, the method comprising: cryopreserving Final NK cells in a cryopreservation media, the Final NK cells comprising less than about 5% of CD3+ cells/WBCs and more than about 90% of CD3-CD56+ cells/WBCs, wherein Final NK are a known HLA-type; and wherein the therapeutic composition comprises more than one concentration of Final NK cells.

In one aspect, the NK cell cryopreservation media comprises, 10% DMSO, and 12.5% (w/v) HSA in Plasmalyte-A.

Also disclosed herein, is a method of treating a recipient subject, the method comprising: determining the recipient subject's HLA-specific antibody profile, excluding a Final NK cell composition that may be cross-reactive with the recipient subject's HLA-specific antibody profile; measuring the recipient subject's weight or body surface area; calculating the total number of NK cells to be administered to the recipient subject; selecting at least one Final NK cell composition according to the total number of NK cells to be administered to the recipient subject; and administering the Final NK cell composition to the recipient subject.

In one embodiment, the recipient subject has a cancer or a tumor.

In another embodiment, the recipient subject has an infectious disease.

According to another non-limiting aspect of the present disclosure, a system for determining the Final NK Cell Composition ID to be administered to a patient, the system comprising: a memory device storing patient data, clinical objective and acceptance data, donor data, and Final NK Cell Composition ID data; a clinical decision algorithm configured to determine the Final NK Cell Composition IDs to be administered to the patient; a processor communicatively coupled to the memory device, the processor in conjunction with the clinical decision algorithm configured to: determine incompatibles between the patient and the Final NK Cell Composition ID, calculate a minimum and maximum NK cell dose range, and determine the Final NK Cell Composition IDs to be administered to the patient.

In one aspect, the patient data includes at least one of a weight in kg, body surface area in m², or a dose of NK cells/kg.

In another aspect, the clinical objective and acceptance data includes at least one of a maximum CD3+ cell dose in cells/kg or a minimum and maximum NK cell dose range in cells/kg.

In one embodiment, the donor data includes at least one of a specimen collection date or an HLA-type.

In another embodiment, the Final NK Cell Composition ID data includes at least one of a total number of CD3-CD56+ cells/mL, a percentage of CD3+ cells/WBCs, or a total storage volume in mL.

### BRIEF DESCRIPTION OF THE DRAWINGS

FIG. 1 shows a flow diagram example of one embodiment, the collection of mononuclear cells (MNCs), CD3-depletion, and the NK cell expansion process.

FIG. 2 shows a representative immunophenotyping analysis (using flow cytometry). Day 13 Expanded NK cells were first gated on single cells (FSC-A vs. FSC-H, not shown). Single cells were gated to remove debris smaller than leukocytes, then WBCs, CD45+(CD45− VivoBlue). Viable WBCs were gated as 7-AAD-negative, then gated for NK cells (CD3−CD56+) and T cells and NK T cells (CD3+). Last, the CD56− cells were gated for feeder cells (CD137L+/CD32+).

FIG. 3 is a diagram of a clinical decision support system according to an example embodiment of the present disclosure.

FIG. 4 is a flow diagram of an example procedure to create the decision algorithms disclosed herein, according to an example embodiment of the present disclosure.

FIG. 5 shows no difference in percent cell viability (FIG. 5A), percent cell recovery (FIG. 5B), and alive cell number (FIG. 5C) of four different concentrations FCs cryopreserved in cryovials. The following four different cryopreservation concentrations were evaluated: 5×10⁶ cells/cryovial, 1×10⁷ cells/cryovial, 2×10⁷ cells/cryovial, and 5×10⁷ cells/cryovial.

FIG. 6 shows no difference in percent cell viability (FIG. 6A) and percent cell recovery (FIG. 6B) of irradiated FCs (IFCs) cryopreserved and stored in cryobags two different concentrations (Low, 0.25×10⁹ cells/cryobag and Medium, 0.5×10⁹ cells/cryobag).

FIG. 7 shows an improvement in percent cell viability of CD3-depleted MNCs cryopreserved and stored in Test In-Process Cryoprotectant Media, comprised of 10% dimethyl sulfoxide (DMSO) and 40% fetal bovine serum (FBS) in 50% RPMI Complete Media with GlutaMAX compared to Control In-Process Cryoprotectant Media, comprised of 10% DMSO, 50% (v/v) human serum albumin (HSA), and 40% Plasmalyte-A. No difference was in percent cell viability was shown.

FIG. 8 shows stability of fresh, Expanded NK cells at room temperature (FIGS. 8A, 8B) and stability of Expanded NK cells following cryopreservation and thaw (FIGS. 8C, 8D) at room temperature.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Definitions

Some definitions are provided hereafter. Nevertheless, definitions may be located in the “Embodiments” section below, and the above header “Definitions” does not mean that such disclosures in the “Embodiments” section are not definitions.

As used herein, the specification and the appended claims, the singular forms “a,” “an,” and “the” include plural referents unless the context clearly dictates otherwise. Thus, for example, reference to “a component” or “the component” includes two or more components.

The term “and/or” used in the context of “X and/or Y” should be interpreted as “X,” or “Y,” or “X and Y.” Similarly, “at least one of X or Y” should be interpreted as “X,” or “Y,” or “X and Y.”

As used herein, “about,” “approximately,” and “substantially” are understood to refer to numbers in a range of numerals, for example the range of −10% to +10% of the referenced number, preferably −5% to +5% of the referenced number, more preferably −1% to +1% of the referenced number.

Ranges can be expressed herein as from “about” or “approximately” one particular value, and/or “about” or “approximately” to another particular value. When such a range is expressed, another embodiment includes from the one particular value and/or to the other particular value. Similarly, when values are expressed as approximations, by use of the antecedent “about” or “approximately,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” or “approximately,” that particular value in addition to the value itself. For example, if the value “10” is disclosed, then “about 10” is also disclosed. It is also understood that when a value is disclosed that “less than or equal to” the value, “greater than or equal to the value,” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The word “increase” can refer to any change that results in a greater amount of a symptom, disease, composition, condition or activity. An “increase” can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

The word “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The words “optional” or “optionally” mean that the subsequently described event or circumstance may or may not occur, and that the description includes instances where said event or circumstance occurs instances where it does not.

The terms “T cells,” “NK T cells,” “CD3+ cells,” and/or “CD3-positive cells” are used interchangeably herein to refer to a type of lymphocyte or immune cell that expresses CD3 proteins.

The term “primers” are a subset of probes which are capable of supporting some type of enzymatic manipulation and which can hybridize with a target nucleic acid such that the enzymatic manipulation can occur. A “primer” can be made from any combination of nucleotides or nucleotide derivatives or analogs available in the art which do not interfere with the enzymatic manipulation.

The term “probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

The terms “peptide,” “polypeptide,” and “protein” are used interchangeably to refer to a polymer of amino acid residues.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

As various changes could be made in the above-described cells and methods without departing from the scope of the invention, it is intended that all matter contained in the above description and in the examples given below, shall be interpreted as illustrative and not in a limiting sense.

The terms “inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “reduce” or other forms of the word, such as “reducing” or “reduction,” refers to the lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “prevent” or other forms of the word, such as “preventing” or “prevention,” is meant to stop a particular event or characteristic, to stabilize or delay the development or progression of a particular event or characteristic, or to minimize the chances that a particular event or characteristic will occur. “Prevent” does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “subject,” “recipient subject,” and/or “patient” are used interchangeably and refer to any individual who is the target of administration or treatment. The “subject” can be a vertebrate, for example, a mammal. In one aspect, the “subject” can be human, non-human primate, bovine, equine, porcine, canine, or feline. The “subject” can also be a guinea pig, rat, hamster, rabbit, mouse, or mole. Thus, the “subject” can be a human or veterinary patient. The term “patient” refers to a “subject” under the treatment of a clinician, e.g., physician, or a healthcare professional.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “administration” to a subject includes any route of introducing or delivering to a subject an agent. “Administration” can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. The terms “concurrent administration,” “administration in combination,” “simultaneous administration,” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. The term “systemic administration” refers to the introducing or delivering to a subject an agent via a route which introduces or delivers the agent to extensive areas of the subject's body (e.g. greater than 50% of the body), for example through entrance into the circulatory or lymph systems. By contrast, the term “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

The terms “treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

The words “comprise,” “comprises,” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the embodiments and methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

Where used herein, the terms “example” and “such as,” particularly when followed by a listing of terms, are merely exemplary and illustrative and should not be deemed to be exclusive or comprehensive.

### EMBODIMENTS

Master Cell Bank (MCB) and Working Cell Banks (WCBs)

In one embodiment, a delivery vehicle, such as feeder cells (FCs) (for example K562 clone CSTX002, expressing membrane-bound IL-21 and 4-1BBL or other activating agents), can be used as artificial antigen presenting cells in a co-culture to expand NK cells. It is understood and herein contemplated that various delivery vehicles may be used to expand NK cells ex vivo. The FCs can undergo compendial testing and/or can be cryopreserved and comprise a Master Cell Bank (MCB). The MCB cells can be stored in cryovials, or any other cryo-storage container, in FC Cryopreservation Media, comprising RPMI Complete Media (with GlutaMAX and 10% fetal bovine serum (FBS)) and 10% dimethyl sulfoxide (DMSO), or any other cell cryopreservation known to a person of skill in the art. The terms “cryoprotectant media,” “cryoprotectant reagent,” “cryopreservation media,” and “cryopreservation reagent” are herein, used interchangeably. Individual Working Cell Banks (WCBs), comprising FCs derived from the MCB, can be generated for routine use and help to maintain the integrity of the MCB. Generally, WCBs can be generated by thawing and washing individual MCB vials, then the FCs can be expanded in RPMI Complete Media with replacement and culture splitting every few days. The FCs can be cryopreserved and stored at a temperature below about −150° C., in liquid or vapor phase nitrogen. The cryopreserved FCs derived from the MCB comprise the WCB. In one embodiment disclosed herein, the WCB comprises FCs in FC Cryopreservation Media within individual cryovials, the quantity of FCs per cryovial is about 2.5×10⁶ cells to about 1×10⁸ cells; preferably, about 1×10⁷ cells to about 2×10⁶ cells.

A controlled rate freezer can be used when cryopreserving a composition containing cells or any other composition that may be damaged by freezing too quickly and/or freezing too slowly and to overcome heat released during phase transition from liquid to solid. A freezing program can be designed to perform the freezing process at a controlled rate to an end temperature and will vary depending on the container and composition. Once the end temperature of the freeze program is reached the composition can be safely and rapidly (or slowly) cooled to the final desired cryopreservation temperature. A freeze curve may also be generated during the freeze program and analyzed after it is complete, to ensure the controlled freeze was successfully performed. For example, in one embodiment, the freezing program for cryopreserving compositions, solutions, and/or reagents may comprise holding at 4.0° C.; Cool at −1.0° C./minute until sample reaches−4.0° C.; Cool at −25.0° C./minute until chamber reaches−40.0° C.; Warm at +10.0° C./minute until chamber reaches−12.0° C.; Cool at 1.0° C./min until chamber reaches−40.0° C.; Cool at −10.0° C./min until Chamber reaches−90.0° C. (end temperature). Verification of successful controlled freeze can be achieved by review of the freeze curve, then the compositions, solutions, and/or reagents can then be cooled to the desired storage temperature, below about −150° C. Additionally or alternatively, any sufficient freezing program or method known to a person of skill in the art may be used.

FCs from the WCB (or the MCB) can be thawed, expanded in culture, irradiated, and used for NK cell expansion. Culture-expanded FCs can be concentrated to about 1×10⁷ cells/mL to about 1×10⁸ cells/mL in RPMI Complete Media, and can be for irradiated for the amount of time necessary to achieve about 100 Gy (or equivalent) central dose. After irradiation, the IFCs can be used for NK cell expansion or can be cryopreserved by diluting the IFCs to a concentration of about 5×10⁶ cells/mL to about 5×10⁷ cells/mL, with FC Cryopreservation Media, and stored in cryobags containing about 20 mL (to about 100 mL (i.e., about 1×10⁸ cells/cryobag to about 5×10⁹ cells/cryobag). The cryobags comprising the IFCs can be cryopreserved and stored at a temperature below about −150° C., in liquid or vapor phase nitrogen. Preferably, a freezer program can be utilized and a freeze curve may be generated and analyzed to verify a controlled freeze was successfully performed.

All cell culture consumables, reagents, media, instruments, etc. should be sterile and all work should be performed in a cell culture hood. Sterile cell culture and aseptic processing techniques as well as current Good Manufacturing Practices (cGMPs) known to a person of skill in the art should be utilized to prevent contamination and control the manufacturing process.

Donor Selection

NK cells are licensed (acquire enhanced killing ability) when they express inhibitory killer immunoglobulin receptors (KIR) for self-HLA class T molecules. This enables NK cells to recognize “self” and spare autologous cells from killing. Targets lacking self-HLA class T molecules are thus more likely to elicit recognition by licensed NK cells. The inhibitory KIR genes known to be relevant for NK alloreactivity are: (i) 2DL1 which binds to HLA-C group 2 alleles, (ii) 2DL2 and 2DL3 which bind to HLA-C group 1 alleles, (iii) and 3DL1 which binds to HLA-B Bw4 alleles. According to the missing-ligand model, for each NK cell expressing an inhibitory KIR gene there will be alloreactive only if the corresponding ligand is absent in the recipient, and present in the donor—e.g., any donor possessing a Group C1 allele is alloreactive to any individual lacking a Group C1 allele. Thus, donors who possess HLA in the C1, C2, and Bw4 families are predicted by this model to be alloreactive against any recipient lacking C1, or C2, or Bw4. In one embodiment, the donor is a mammal. The donor is preferably human.

Whereas inhibitory KIRs prevent alloreactivity, activating KIRs recognize activating ligands that promote NK cell lysis. Inheritance of activating KIR is widely variable-0 to 7 activating KIRs are possible in any one individual. Data from patients undergoing stem cell transplantation show that patients receiving allografts from donors with more activating KIRs have a better outcome than patients receiving allograft from donors with fewer activating KIR. Others have shown a protective benefit against leukemia in individuals that inherit more activating KIRs. The laboratory has shown that NK cells with higher numbers of activating KIR induce stronger lysis of target cells. In addition, the activating KIR 2DS1 and 3DS1 are associated with disease-free survival in multivariate analysis.

NKG2C is an activating receptor that is expressed late in NK cell development and recognizes HLA-E rather than -B or -C. NKG2C expression is induced in patients with CMV infection and correlates with an adaptive NK cell phenotype and improved leukemia-free survival.

Generally, the “optimal” donor is one who has an HLA genotype carrying C1, C2, and Bw4 alleles, has a KIR genotype possessing the inhibitory KIR (2DL1, 2DL2 or 3, and 3DL1) that bind to C1, C2, and Bw4 (leading to maximum licensing), has a high proportion of activating KIR (≥3 of the variably-inherited activating genes including 2DS1 and 3DS1), and has been exposed to CMV resulting in high NKG2C expression.

Considering data available for Caucasian donors, C1/C2/Bw4 alleles occur in 32% of the population. Of the 23 KIR genotypes that account for 80% of the population, 25.3% meet all of these criteria. Approximately 90% of adults have been exposed to CMV. Thus, the “ideal” NK cell donor can be identified in approximately one out of sixteen healthy individuals. It is generally understood and herein contemplated that by screening for and/or selecting donor NK cells from this 1 out of 16 healthy individuals, a “universal” donor NK cell can be obtained that are histologically optimized for at least 50%-85% of recipient subjects.

Accordingly, in one aspect, disclosed herein are methods of selecting universal donor NK cells for therapeutic administration to a recipient subject in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4 and (c) selecting the candidate NK cells as a universal donor NK cell for the therapeutic administration when (i) the HLA genotype indicates the presence of at least two HLA alleles HLA C1, C2, and Bw4 and/or (ii) the KIR genotype indicates the presence of at least three activating KIRs 2DS1/2, 2DS3/5, 3DS1, and/or 2DS4.

Also disclosed herein are methods of screening a population of candidate NK cells from a donor to identify universal NK donor cells in the population for providing a source of NK cells for therapeutic administration to recipient subjects in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2 or 2DL3, and/or 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; wherein candidate NK cells comprising (i) at least two HLA alleles HLA C1, C2, and Bw4 and therefore comprising at least one of the variably inherited inhibitory KIRs 2DL1, 2DL2 or 2DL3, and/or 3DL1 and/or (ii) at least three activating KIRs 2DS1/2, 2DS3/5, 3DS1, and/or 2DS4 are universal NK cells.

In one aspect, disclosed herein are methods of screening a population of NK cells or methods of selecting universal donor NK cells for therapeutic administration to a recipient subject of any preceding aspect, wherein the selected universal donor NK cells are histologically optimized for at least 50%-85% of recipient subjects.

As noted above, NKG2C is an activating receptor that is expressed late in NK cell development and recognizes HLA-E rather than -B or -C. NKG2C expression is induced in patients with CMV infection and correlates with an adaptive NK cell phenotype and improved leukemia-free survival. Thus, identifying candidate donor cells from individuals with elevated NKG2C or that are seropositive for CMV, can further increase the efficacy of the donor NK cells. Thus, also disclosed herein are methods of screening a population of NK cells or methods of selecting universal donor NK cells for therapeutic administration to a recipient subject, where the method further comprises obtaining or having obtained the CMV seropositivity of the candidate NK cells; and wherein the NK candidate NK cells are further selected when the NK cell donor is seropositive for CMV or the NK cells from the NK cell donor have high NKG2C expression compared to a reference level of NKG2C expression.

It is understood and herein contemplated that the disclosed methods of screening and selecting may produce an isolated universal donor NK cell. Accordingly, disclosed herein are isolated universal donor NK cells wherein the isolated universal donor NK cells comprise at least two HLA alleles HLA C1, C2, and Bw4; and/or at least three activating KIRs 2DS1/2, 2DS3/5, 3DS1, and/or 2DS4. In one aspect, the isolated universal donor NK cells are NKG2C+ or derived from a CMV seropositive donor source.

It is further understood that rather than selecting or screening for the candidate donor NK cells from a donor source to obtain universal donor NK cells with the correct genotype features, NK cells or cell lines can engineered to encode and express HLA alleles indicative of the presence of one or more variably inherited inhibitory KIRs 2DL1, 2DL2, 2DL3, and/or 3DL1 and activating KIRs 2DS1/2, 2DS3/5, 3DS1, and/or 2DS4. Accordingly, disclosed herein are engineered NK cells or cell lines, wherein the NK cells have been transformed to express one, two or more HLA alleles comprising C1, C2 or Bw4 (for example an NK cell or cell line that expresses C1, C2, and Bw4) and/or transformed to express of one, two, three, four, five or more variably inherited activating KIRs comprising 2DS1/2, 2DS3/5, 3DS1, or 2DS4.

Though not required, for maximum cost savings and time efficiency, donors can be screened in step-wise algorithm excluding donors from further testing who do not meet criteria.

KIR genotyping can be first performed for NK cell donors with reverse sequence-specific oligonucleotide (SSO) methodology (e.g., One Lambda), including discrimination of Functional vs. Deletion variants of KIR2DL4. KIR-B content can be determined using the B Content Calculator maintained by EMBL-EBI. Activating KIR content can be determined by scoring the total number of activating KIR genes. All DS-designated KIR and Functional KIR2DL4 are considered activating. Donors can be selected who have the common activating KIRs (KIR2DS4 and the functional version of KIR2DL4) and a high number of the five variably-inherited activating KIRs. Donors can also be selected on based on the number of B-KIR segments inherited (e.g., three or four of the centromeric and telomeric B alleles).

NK cell donors can next be HLA typed at intermediate or high-resolution level for alleles at HLA-B and -C loci by SSO-PCR (amplification and oligonucleotide sequencing) using commercial kits. KIR-ligand class can be predicted using the KIR Ligand Calculator maintained by the European Bioinformatics Institute of the European Molecular Biology Labs (EMBL-EBI). Individuals possessing all three C1, C2, and Bw4 classes are selected.

Donors can lastly be tested for CMV. CMV+ donors can be tested to confirm the presence of NKG2C+ NK cells. Alternatively, donors can be screened for the presence of NKG2C+ NK cells above the threshold (e.g., about 20%) that predicts prior CMV exposure.

It is understood and herein contemplated that an NK cell donor can be tested to obtain the donor's HLA genotype and therefore, the donor NK cells' HLA genotype. Methods of testing to obtain the donor's HLA genotype can include any method known to a person of ordinary skill in the art.

The Final NK cell composition can be manufactured prior to patient need. Donors can undergo testing to obtain the donor's HLA genotype as well as standard infectious disease screening and other donor screening (as required by 21 CFR 1271 subpart C) before or after collection. Source PBMCs or MNCs can be collected and NK cells can be expanded. In one embodiment, MNCs are depleted of CD3+ cells using MACS colloidal super-paramagnetic CD3 MicroBeads. The resulting CD3-depleted cells are co-cultured with IFCs and/or membrane particles in media supplemented with fetal calf serum and stimulated with IL-2. After about seven days, the cultures can be restimulated. The final NK cell product can undergo lot release testing and cryopreservation on about day 14 for subsequent infusion. NK cells can be cryopreserved in various concentrations, such as single-dose aliquots (e.g., about 50 mL containing about 1×10⁸ NK cells/mL). Assuming an initial donor blood draw equivalent to 1 unit (450 mL), a median content of about 1.26×10⁵ Final NK cells/mL, and a median expansion of about 2,800-fold in about 2 weeks, each donor can generate sufficient NK cells for about 31 unit-dose cryobags. Assuming an initial donor apheresis containing a median of about 3×10⁸ NK cells after CD3-depletion, each donor can generate an average of about 168 unit-dose cryobags. One cryobag of the Final NK cell composition, containing about 5×10⁹ Final NK cells, can be sufficient for about one dose of 1×10⁸ Final NK cells/kg for a 50 kg individual. Doses of about 1×10⁸/kg can therefore require up to 2-3 cryobags per patient per dose for adult patients. Assuming freezing media containing 10% DMSO, the DMSO administered for a 10⁸/kg dose will be 0.1 mL/kg. When taking into account patient (recipient subject) variability, for example, size differences (i.e., weight or body surface area), it may be more efficient to have at least more than one concentration of cryopreserved Final NK cells within the Final NK cell compositions. Thus, in order to administer one dose of 1×10⁸ Final NK cells/kg to a smaller recipient subject, such as a child of about 16 kg, approximately 68% of the Final NK cell composition would be in excess of the desired dose and would be removed and discarded after thawing, if the cryobag contained 5×10⁹ Final NK cells. Thus, importantly, it is understood and herein contemplated that the Final NK cell composition may comprise more than one cryopreserved concentration of Final NK cells; preferably, more than four.

Isolation of Mononuclear Cells (MNCs)

MNCs can be isolated from donor whole blood or buffy coat products, removing unwanted red blood cells (RBCs) and granulocytes. Specimens containing about 10 mL to about 450 mL can be transported at a controlled room temperature of about 15° C. to about 30° C. and preferably processed within about 48 hours. Low speed centrifugation of a donor whole blood or buffy coat product on a cushion of Ficoll™ density gradient solution can allow separation of light density cells, which includes all mononuclear cells (lymphocytes, monocytes, and blasts) from higher density cells such as RBCs and granulocytes. This can be achieved for example, by various methods known a person of skill in the art including, but not limited to: (1) manually layering blood or buffy coat on top of a Ficoll™ solution in a standard conical tube; (2) using SepMate™ conical tubes which minimize mixing of the specimen and Ficoll™; or (3) using VACUTAINER® CPT™ Cell Preparation Tubes with Sodium Citrate. Isolated MNCs can then be washed to remove residual Ficoll™ or other solution and to provide cells with an appropriate media or buffer for further processing.

MNCs can also be isolated from a donor via apheresis. Apheresis collection of MNCs from unmobilized donors can process about 2 to about 6 times the donor blood volume by following standard clinical procedures. Specimens containing about 50 mL to about 500 mL can be transported at a controlled room temperature of about 15° C. to about 30° C. It is preferred that specimens that are transported and/or stored overnight, be under refrigerated conditions of about 2° C. to about 10° C. These specimens transported and/or stored overnight are preferably diluted in donor plasma, or any acceptable buffer solution containing 0.5% (w/v) of HSA, to a concentration below about 2×10⁸ cells/mL. Specimens are preferably processed within about 48 hours.

In another embodiment, specimens can be processed for buffy coat by whole blood centrifugation followed by removal of plasma and collection of the white blood cell-rich interface (i.e., buffy coat), leaving behind the majority of RBCs. Processing specimens for buffy coat is typically only necessary when whole blood collections are greater than about 300 mL, resulting in a corresponding buffy coat of about 25 mL to about 250 mL.

The isolated donor MNCs can be cryopreserved and stored or can be used for NK cell expansion and/or undergo T cell-depletion. In one embodiment of the present disclosure, the MNCs may be diluted in an In-Process Cryoprotectant Media to a volume of about 25 mL at a concentration of about 5×10⁶ cells/mL to about 200×10⁶ cells/mL, preferably about 50×10⁶ cells/mL to about 100×10⁶ cells/mL, for storage in about 25 mL in cryobags or in about 1.5 mL in cryovials. The In-Process Cryoprotectant Media can comprise, 10% DMSO and 40% FBS in 50% RPMI Complete Media with GlutaMAX or the In-Process Cryoprotectant Media may comprise of 10% DMSO, 6.25% (w/v) HSA, and 40% Plasmalyte-A. Additionally or alternatively, the In-Process Cryoprotectant Media can comprise any cryoprotectant media known to a person of skill in the art. The MNCs can be cryopreserved and stored at a temperature below about −150° C., in liquid or vapor phase nitrogen. Preferably, a freezer program can be utilized and a freeze curve may be generated and analyzed to verify a controlled freeze was successfully performed.

Surprisingly, In-Process Cryoprotectant Media comprising 10% DMSO, 40% FBS, and 50% RPMI Complete Media, in combination with decreasing the target volume prior to cryopreservation, for example, decreasing the target volume from about 25 mL to about 1.5 mL (i.e., using a smaller cryo-storage container), improved the post-thaw viable MNC recoveries.

Therefore, it is understood and herein contemplated that if MNCs are cryopreserved, it is preferable to concentrate the MNCs for cryopreservation with In-Process Cryoprotectant Media comprising, 10% DMSO, 40% FBS, and 50% RPMI Complete Media, to a volume of about 0.5 mL to about 5 mL, preferably about 1.0 mL to about 1.8 mL with a concentration of about 50×10⁶ cells/mL to about 100×10⁶ cells/mL, and store in cryovials, or an equivalent sized cryo-storage vial or tube.

In another aspect of the present disclosure, isolated donor MNCs can be cryopreserved in a commercially available, chemically defined cryoprotectant solution, such as STEM-CELLBANKER™ (Amsbio). STEM-CELLBANKER™ reportedly contains 5-10% DMSO and ≤10% inorganic salts and has been optimized for cryopreservation directly at about −80° C., without the need for a controlled rate freezer.

MNCs that are not cryopreserved can be stored at about room temperature for up to about 48 hours before proceeding to NK cell expansion or T cell-depletion.

NK Cell Expansion and T Cell-Depletion Methods

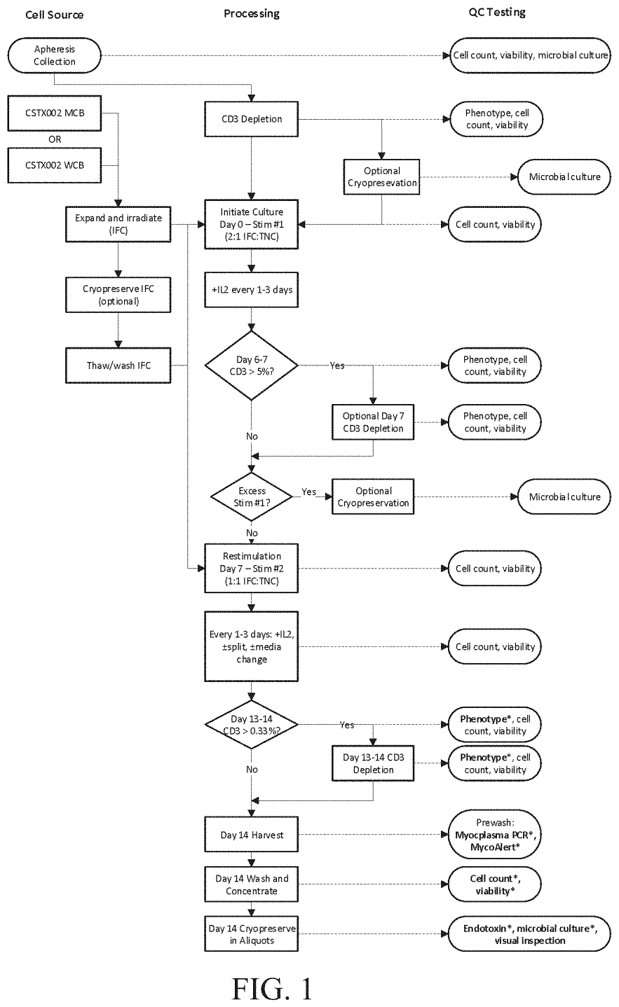

NK cells can be expanded from isolated, enriched MNCs to achieve high numbers for use as potent anti-tumor or anti-viral therapeutic agents and can also be used along with chemotherapies, monoclonal antibody therapies, other immunotherapies, stem cell transplants, or any other therapy or treatment known to a person of skill in the art. One embodiment of the NK cell expansion process can be visualized in FIG. 1.

It is understood and herein contemplated that the starting product (i.e., cells) to be activated and expanded (NK expansion), can include donor cells from a healthy donor, an unhealthy donor, a universal donor, an optimal donor, an engineered universal donor, and/or cell lines. It is also herein understood, that expansion in the presence of one or more NK cell activating agents (e.g., stimulatory peptides, cytokines, aptamers, and/or adhesion molecules) can help to proliferate NK cells and also helps to overcome many hurdles associated with cytokine toxicity.

MNC enrichment may not be necessary post-collection of whole blood or buffy coat prior to depletion of CD3+ cells (i.e., T cells and/or NK T cells).

Disclosed herein are methods of depleting MNCs of T cells, wherein the T cells can be labeled with antibodies specific to T cell markers, followed by removal of the magnetically- or fluorescently-labelled cells by passage through a magnetic field or fluorescence activated cell sorter, respectively. Generally, T cells can be labeled with CD3 markers, but other T cell or leukocyte markers that are absent from NK cells, including, but not limited to CD4, CD8, CD1, CD13 or other markers known to one of skill in the art may be used. Additionally or alternatively, T cell or leukocyte markers may be used in combination to deplete T cells from MNC compositions. The terms “T cell-depletion” and “CD3-depletion” are used interchangeably herein.

In one aspect, it is preferred to T cell-deplete MNCs prior to initiating the NK cell expansion process. Up-front reduction of CD3+ cells not only encourages more rapid NK cell expansion, but also minimizes negative side effects, such as Graft-Versus Host Disease (GVHD), from allogeneic T cells. However, in-process CD3-depletion may be performed. In addition or alternatively, CD3-depletion may be performed more than once.

In another embodiment, large-scale starting cell products such as MNC apheresis collections, can be T cell-depleted using a functionally-closed clinical-grade cell sorter capable of processing 1-100 billion cells, such as Miltenyi Biotec's CliniMACS Plus®, CliniMACS Prodigy®, or MACSQuant® Tyto® devices.

For smaller scale starting cell products, such as MNC preparations from whole blood, CD3-depletion can be accomplished by using a research-grade magnetic cell capture system, for example, the Miltenyi SuperMACS® magnet system with XS column. CD3+ cells can be captured in the XS column. Due to the open nature of such systems, it is preferred that T cell-depletion be performed in a classified clean room. Many research-grade systems can process 1×10⁶ cells to 2×10¹⁰ cells in a single run.

Reagents and solutions that may be used during the NK cell expansion process can be prepared ahead of time. For example, about 25 mL aliquots comprising a high quality (HQ) FBS, such as Hyclone FBS (heat inactivated and irradiated) can be prepared in about 50 mL sterile tubes. If not used within about 24 hours, the HQ FBS aliquots can be stored at about −80° C. If not used within about 1 or 2 hours but less than about 24 hours, the HQ FBS aliquots can be kept on ice or stored at 4° C. If used within about 1 or 2 hours, the HQ FBS aliquots can be kept at about room temperature. About 5 mL aliquots comprising GlutaMAX can be prepared in about 15 mL sterile conical tubes and stored at about room temperature. IL-2 Stock Solution can be prepared, comprising about 22×10⁶ U IL-2 in about 10 mL sterile water. About 120 μL aliquots of the IL-2 Stock Solution can be prepared in sterile vials or microfuge tubes. If not used within about 24 hours, the IL-2 Stock Solution aliquots can be stored at about −80° C. If not used within about 1 or 2 hours but less than about 24 hours, the IL-2 Stock Solution aliquots can be kept on ice or stored at 4° C. If used within about 1 or 2 hours, the IL-2 Stock Solution aliquots can be kept at about room temperature.

NK cell expansion can be performed using G-Rexx devices, or any other cell culture flask or device known to a person of ordinary skill in the art.

RFG-NK Expansion Media can be prepared by adding about 50 mL of HQ FBS and about 5 mL GlutaMAX to about 500 mL sterile RPMI Complete Media (RPMI 1640 Complete Media with Phenol Red, and without L-glutamine). The RFG-NK Expansion Media can be stored at approximately 2° C. to about 8° C. until needed. Prior to use during NK expansion, the RFG-NK Expansion Media should be transferred to an incubator or water bath at about 37° C. for a minimum of about 15 minutes.

Freshly prepared IL-2 Stock Solution aliquots or recently thawed IL-2 Stock Solution aliquots can be used to prepare an IL-2 Working Solution comprising about a 1:100 dilution of IL-2 Stock Solution in RFG-NK Expansion Media; the IL-2 Working Solution comprising about 2×10⁴ U IL-2 per mL of RFG-NK Expansion Media.

Various cell culture devices sizes can be used to culture and expand NK cells, such as the following G-Rex® devices, G-Rex10, G-Rex10M, G-Rex100, G-Rex100M, and/or G-Rex500M. Additionally or alternatively, other cell culture devices generally known to a person of skill in the art may be used. Table 1 provides an example of volumes and number of cells that can be used with optional cell culture device sizes for co-culture initiation of Stimulation 1.

TABLE 1
Co-Culture Initiation of Stimulation 1 in Four Different
G-Rex ® Cell Culture Devices

| G-Rex ® Cell | | | | |
|---|---|---|---|---|
| Culture Device | G-Rex10 | G-Rex10M | G-Rex100 | G-Rex100M |
| Surface Area | 10 | cm² | 100 | cm² |
| Initial RFG-NK | 10 | mL | 100 | mL |
| Expansion Media Added | | | | |
| Total Fill Volume | 40 mL | 100 mL | 450 mL | 1,000 mL |
| IL-2 Working Solution | 0.2 mL | 0.5 mL | 2.3 mL | 5.0 mL |
| volume (2 × 10⁴ U/mL; | | | | |
| 100 U/mL final) | | | | |

| | | | |
|---|---|---|---|
| Preferred Minimum Seed | >100 | MNCs | 20 × 10⁶ MNCs |
| (No. MNCs/G-Rex ®⁾ | | | |
| Optimum Seed (No. | 4 × 10⁶ | MNCs | 40 × 10⁶ MNCs |
| MNCs/G-Rex ®) | | | |
| Preferred Maximum Seed | 5.8 × 10⁶ | MNCs | 58 × 10⁶ MNCs |
| (No. MNCs/G-Rex ®) | | | |

Co-culture initiation of Stimulation 1 can begin by adding the initial volume of RFG-NK Expansion Media into the G-Rex® devices. For example, about 10 mL of RFG-NK Expansion Media for devices with a surface area of about 10 cm² and about 100 mL of RFG-NK Expansion Media for devices with a surface area of about 100 cm². Next, an appropriate volume of the IL-2 Working Solution can be added to the cell culture device such that the final concentration of IL-2 will be approximately 100 U/mL. Next, the desired number of MNCs can be added to the cell culture device. For example, more than about 100 MNCs to about 5.8×10⁶ MNCs, preferably about 4×10⁶ MNCs, for devices with a surface area of about 10 cm² and about 20×10⁶ MNCs to about 58×10⁶ MNCs, preferably about 40×10⁶ MNCs, for devices with a surface area of about 100 cm². All MNCs from the same donor specimen, that begin NK expansion (culture day 1) on the same date, can be identified by a unique batch number that is that is compliant with internationally recognized coding and labelling systems for blood, blood derivatives, tissues and cell therapies. This unique batch number is carried throughout the expansion process such that the Final NK cell compositions can also be identified by the batch number, and is comprised of 13 characters—the first 5 uniquely identify the Ohio State University Cell Therapy Laboratory, the next 2 refer to the year, and the last 6 are assigned sequentially.

The IFCs can be thawed in a water bath or bead bath at a temperature of about 37° C., diluted in about 25 mL to about 40 mL of RFG-NK Expansion Media (previously warmed to about 37° C.) and washed by centrifugation at about 900×g for about 10 minutes at about room temperature. After a second wash, the RFG-NK Expansion Media can be removed by aspiration and the pellet comprising the IFCs can be resuspended in about 20 mL of RFG-NK Expansion Media. A sample of resuspended IFCs can be removed and assessed for number of viable IFCs. IFCs can be added to individual cell culture devices. The ratio of IFCs to MNCs can be about 1:1 to about 3:1. Preferably, the ratio of IFCs to MNCs for NK expansion should be about 2:1. The volume of additional RFG-NK Expansion Media can be added to the cell culture devices to bring the volume up to the total fill volume. The cell culture devices can be placed in an incubator, at about 37° C. and about 5% CO₂.

It is understood and herein contemplated that activating agents (e.g., IL-2, IL-21, and/or 4-1BBL) may be delivered to the culture during the NK cell expansion process by directly adding them to the cell culture media at appropriate times and/or via a delivery vehicle, such as FCs or microparticles. It is understood and herein contemplated that one activating agent may be used during NK cell expansion. Preferably, more than one activating agent may be used.

During about the first week of NK expansion, Simulation 1 can be supplemented or replenished with additional IL-2 about every 2 to 3 days. During IL-2 supplementation, a fresh IL-2 Working Solution can be prepared, comprising about 2×10⁴ U IL-2 per mL RFG-NK Expansion Media. The IL-2 Working Solution (warmed to approximately 37° C.) can be added to the individual cell culture devices such that the final concentration of supplemented IL-2 will be approximately 100 U/mL.

During NK expansion, small samples, for example about 1 mL to about 3 mL of media and cells can be removed from individual cell culture devices in order to evaluate the number and phenotype of the cells during the NK expansion process. For example, the number of viable cells can be assessed by using a Trypan Blue Viability Assay or any other method of cell viability known to a person of skill in the art (i.e., the number of live cells/number of total cells (live+dead)×100). In addition or alternatively, an immunophenotype analysis may be performed by Flow Cytometry of any other method of phenotyping immune cells generally known to a person of skill in the art. Performing an immunophenotype analysis and determining the number of viable cells can be used to determine the whether a subsequent CD3-depletion is required. CD3-depletion may be performed once or more than once.

At about day seven (i.e., the end of Stimulation 1), the cells may be harvested for re-stimulation or can undergo CD3-depletion followed by re-stimulation or cryopreservation and storage. The cells may be evaluated for cell viability and immunophenotype, as previously described. Generally, if the immunophenotypic analysis reveals more than about 5% CD3+ cells, it is preferable to perform T cell-depletion at this time.

Without mixing or significantly disturbing the cells on the bottom of the membrane or the bottom of the cell culture device, the spent media from the top surface may be carefully removed or aspirated. The volume of spent media removed can be, for example, about 30 mL to about 90 mL of spent media removed for devices with a surface area of about 10 cm² and about 400 mL to about 900 mL of spent media removed for devices with a surface area of about 100 cm². Next, the remaining cell suspension within the cell culture device can be transferred equally, into one or more than one conical tube. The conical tubes can be centrifuged at about 900×g for about 10 minutes at about room temperature. After centrifugation, the supernatant may be aspirated and the pellets may be resuspended in about 30 mL to about 50 mL of RFG-NK Expansion Media (previously warmed to approximately 37° C.), or if CD3-depletion is to be performed at this time, the pellets may be resuspended in about 30 mL to about 50 mL of CD3-Depletion Buffer (previously warmed to approximately 37° C.), such as CliniMACS® Buffer. It is also understood that at this time, cell viability may be assessed (as previously described) to determine the approximate number of viable cells at the end of Stimulation 1.

The number of viable cells present at the conclusion of Stimulation 1 can be used when calculating the number of Stimulation 1 cells (Stim1 cells) to be seeded in the cell culture devices for co-culture initiation of Stimulation 2 (See Table 2).

TABLE 2
Co-Culture Initiation of Stimulation 2 in Four Different
G-Rex ® Cell Culture Devices

| G-Rex ® Cell | | | | |
|---|---|---|---|---|
| Culture Device | G-Rex10 | G-Rex10M | G-Rex100 | G-Rex100M |
| Surface Area | 10 | cm² | 100 | cm² |
| Initial RFG-NK | 10 | mL | 100 | mL |
| Expansion Media Added | | | | |
| Total Fill Volume | 40 mL | 100 mL | 450 mL | 1,000 mL |
| IL-2 Working Solution | 0.2 mL | 0.5 mL | 2.3 mL | 5.0 mL |
| volume (2 × 10⁴ U/mL; | | | | |
| 100 U/mL final) | | | | |

| | | | |
|---|---|---|---|
| Preferred Minimum Seed | >100 | Stim1 cells | 100 × 10⁶ Stim1 cells |
| (No. Stim1 cells/G-Rex ®) | | | |
| Optimum Seed (No. | 20 × 10⁶ | Stim1 cells | 200 × 10⁶ Stim1 cells |
| Stim1 cells/G-Rex ®) | | | |
| Preferred Maximum Seed | 29 × 10⁶ | Stim1 cells | 288 × 10⁶ Stim1 cells |
| (No. Stim1 cells/G-Rex ®) | | | |

Co-culture initiation of Stimulation 2 can begin by adding the initial volume of RFG-NK Expansion Media into the G-Rex® devices. For example, about 10 mL of RFG-NK Expansion Media for devices with a surface area of about 10 cm² and about 100 mL of RFG-NK Expansion Media for devices with a surface area of about 100 cm². Next, an appropriate volume of fresh IL-2 Working Solution (previously warmed to approximately 37° C.) can be added to the cell culture device such that the final concentration of IL-2 will be approximately 100 U/mL. Next, the Stim1 cells remaining from Stimulation 1 can be added to the cell culture device. For example, more than about 100 Stim1 cells to about 29×10⁶ Stim1 cells, preferably about 20×10⁶ Stim1 cells, for devices with a surface area of about 10 cm² and about 100×10⁶ Stim1 cells to about 288×10⁶ Stim1 cells, preferably about 288×10⁶ Stim1 cells, for devices with a surface area of about 100 cm². The desired number of IFCs can be added to individual cell culture devices at a ratio of IFCs to Stim1 cells at about 0.5:1 to about 3:1, preferably at about 1:1. The volume of additional RFG-NK Expansion Media can be added to the cell culture devices to bring the volume up to the total fill volume. The cell culture devices can be placed in an incubator, at about 37° C. and about 5% CO₂.

Additionally or alternatively, co-culture initiation of Stimulation 2 can begin by seeding the Stimulation 1 NK cells harvested on day 5, 6, 7, or 8 at an initial concentration of about 0.5×10⁶ cells/mL to about 1.0×10⁶ cells/mL in RFG-NK Expansion Media in a bioreactor such as the Xuri™ Cell Expansion System. Next, an appropriate volume of fresh IL-2 Working Solution (previously warmed to approximately 37° C.) can be added to the bioreactor such that the final concentration of IL-2 will be approximately 100 U/mL. The desired number of IFCs can be added at a ratio of IFCs to Stim1 cells at about 0.5:1 to about 3:1, preferably at about 1:1. The amount of aeration and mixing can controlled by adjusting the rocking angle from about 4° to about 12° and the rocking speed from about 2 rpm to about 12 rpm. The bioreactor can be set at about 37° C. and about 5% CO₂, with measurement and/or control of pH or dissolved oxygen.

During Stimulation 2, cells can be supplemented or replenished with IL-2 as previously described, about every 1 to 3 days. For cell culture devices, such as G-Rex10 and G-Rex100, but generally not for G-Rex10M and G-Rex100M, about at least half of the cell culture device's media can be exchanged about every 1 to 3 days if a splitting is not being performed at that time. If a bioreactor is used, fresh media can be added to the bioreactor until maximum volume is met, and then perfusion can begin by removing spent media and replacing with fresh media in about 50 to about 500 mL aliquots.

During Stimulation 2, cells may be monitored for cell viability (as previously described) about every 1 to 3 days if desired, to determine the approximate number of viable cells and to determine whether splitting the culture is necessary. If desired, immunophenotype analysis, as previously described, may also be performed at this time. Generally, if the immunophenotypic analysis reveals more than about 5% CD3+ cells, it is preferable to perform T cell-depletion at this time.

In an effort to prevent unwanted overgrowth of cells during Stimulation 2 of the NK expansion, cultures can be split into one or more additional cell culture devices to result in a total number of cells in a cell culture device to about less than 10×10⁶ cells/cm². For example, less than about 1,000×10⁶ cells per GRex® cell culture devices or other cell culture devices with a surface area of about 10 cm². If a bioreactor is being used, total culture volume and perfusion rate can be used to maintain a cell concentration of about 0.5×10⁶ cells/mL to about 1.5×10⁶ cells/mL.

If it has been determined that splitting is necessary, the number of additional cell culture devices needed can be calculated. The volume of media can be reduced first by removal of the spent media without mixing or disturbing the cells on the bottom of the membrane or the bottom of the cell culture device. After resuspending the cells in the residual media, the appropriate volume of residual media and cells can be removed from each cell culture device and “split” or distributed evenly into new cell culture devices. Lastly, IL-2 and RFG-NK Expansion Media can be added to bring the volume up to the total fill volume of each cell culture device. Thus, the original contents of each cell culture device that required splitting will now be distributed among an appropriate number of new cell culture devices with about 5×10⁶ cells/cm² to about 10×10⁶ cells/cm² (G-Rex100 device and G-Rex100M devices) present in each new cell culture device. For example, a G-Rex100M device containing about 2800×10⁶ cells can be split at approximately 1:3 by transferring about ⅓ of the residual volume to two new G-Rex100M devices, and bringing all three devices back up to about 1000 mL, with each device seeded with about 2800±3=933×10⁶ cells or about 9.33×10⁶ cells/cm².

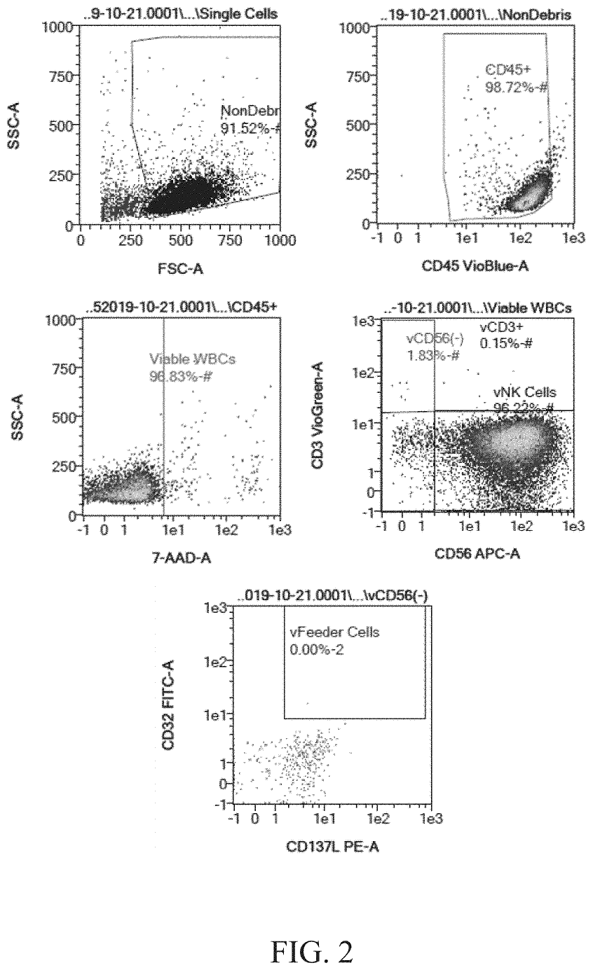

Cells may be analyzed for cell viability and immunophenotype at any point during the NK expansion process. Additionally or alternatively, cells may be analyzed for cell viability and immunophenotype more than once during the NK expansion process. After the cells have been in Stimulation 2 co-culture for more than about 12 days but less than about 18 days, preferably about 13 to about 14 days, cell viability and immunophenotype can be determined. Any cell viability method known to a person of skill in the art may be used to analyze the number of viable cells. In addition, any immunophenotyping method known to a person of skill in the art may be used to determine the immunophenotype of the cells in culture. Preferably, the multiparameter immunophenotyping analysis performed can determine a concentration (per mL) and/or percentage of WBCs (CD45+) that are T cells (CD3+), Natural Killer T cells (NK-T cells) (CD3+CD56+), NK cells (CD3-CD56+), and FCs (CD56-CD32+CD137L+) present in the culture. A sufficient number of cells/events can be analyzed to allow detection of rare cells, typically about 500,000 to about 1,000,000 viable CD45+ cells. FIG. 2 shows a representative immunophenotyping analysis (using flow cytometry).

In-process T cell-depletion can be performed at any point during the NK cell expansion process. Preferably, T cell-depletion is performed on MNCs prior to expansion. Additionally or alternatively, T cell-depletion can also be performed for the first time during NK expansion. In-Process T cell-depletion can be performed at any point during the NK cell expansion process, on cells that have been CD3-depleted at least once; preferably, the subsequent in-process CD3-depletion is performed after about one week of NK cell expansion. Generally, T cell-depletion is preferably if the immunophenotype of the cells that have been in culture for about one week, comprises more than about 5% CD3+ cells. After about day 11 or about day 13, T cell-depletion can be performed if the immunophenotype of the cells in culture comprises more than about 5% CD3+ cells, more than about 1% CD3+ cells, or more than about 0.33% CD3+ cells. Following CD3-depletion, the cells can be cryopreserved and stored as an in-process intermediate and can continue expanded at a later time or can continue the expansion process immediately. If cryopreserved, the in-process intermediate can be cryopreserved and stored in an In-Process Cryoprotectant Media, comprised of 10% DMSO, 6.25% (w/v) HSA, and 40% Plasmalyte-A. Preferably, the In-Process Cryoprotectant Media is comprised of 10% DMSO and 40% FBS in 50% RPMI Complete Media with GlutaMAX. The in-process intermediate can be cryopreserved at a range of 5×10⁶ cells/mL to about 200×10⁶ cells/mL; preferably about 50×10⁶ cells/mL to about 100×10⁶ cells/mL in cryovials with about a 0.5 mL to about 1.8 mL volume capacity, at a temperature below about −150° C., in liquid or vapor phase nitrogen. Preferably, a freezer program can be utilized and a freeze curve may be generated and analyzed to verify a controlled freeze was successfully performed.

In another aspect of the present disclosure, the in-process intermediates can be cryopreserved in a commercially available, chemically defined cryoprotectant solution, such as STEM-CELLBANKER™ (Amsbio). STEM-CELLBANKER™ reportedly contains 5-10% DMSO and ≤10% inorganic salts and has been optimized for cryopreservation directly at about −80° C., without the need for a controlled rate freezer.

The cryopreserved, CD3-depleted in-process intermediate can be thawed, for example in water bath or bead bath at a temperature of about 37° C. and diluted approximately 1:5 with In-Process Thaw Media at about room temperature. In one embodiment, the In-Process Thaw Media is comprised of RFG-NK Expansion Media and 50 U/mL DNase with 400 mg/mL MgCl₂, with the RFG-NK Expansion Media comprised of RPMI Complete Media with GlutaMAX and 10% FBS. The CD3-depleted in-process intermediate can be washed in In-Process Thaw Media by centrifuging at about 300×g for approximately 10 minutes at about room temperature with about 50% brake applied. The supernatant can be removed and the pellet can be resuspended with about 10 mL of In-Process Thaw Media for CD3-depleted in-process intermediates from cryovials and about 70 mL of In-Process Thaw Media for CD3-depleted in-process intermediates from cryobags and washed a second time. The supernatant from the washed CD3-depleted in-process intermediate can be removed and the pellet can be resuspended with about 10 mL of In-Process Thaw Media. Following the washes, the CD3-depleted in-process intermediate can be re-seeded for continued NK cell expansion as previously described.

Expanded NK cells can be harvested after Stimulation 2, after about 12 days to about 18 days, preferably approximately 2 weeks in culture. If necessary or desired, the cells can continue the NK cell expansion process by going through a third stimulation and up to another week of co-culture. To harvest the NK cells, the spent media can be removed by aspirating the media from the top surface, without mixing or disturbing the cells on the bottom of the membrane or the bottom of the cell culture device. A sample of the removed spent media can be assessed for contamination such as mycoplasma, by any method known to a person of skill in the art.

The spent media remaining in the cell culture device and the cells can be thoroughly mixed in each cell culture device by thoroughly mixing or swirling and/or pipetting. All cells can then be transferred into one or more sterile conical tubes. Additionally or alternatively, NK cells can be harvested at multiple time points during the second and third week of a perfusion bioreactor culture in a more efficient “continuous culture” mode. For example, once a steady growth rate is achieved, removal of one-half of the culture (media and cells) and replacement with fresh media at a frequency equal to the doubling time can result in no change in growth rate over several days or even weeks.

The cell suspension comprising the expanded NK cells can concentrated by centrifugation at about 900×g for about 10 minutes at about room temperature. Following centrifugation, the remaining spent media can be removed or aspirated from the tube. The pellet comprising cells can be resuspended in NK Cell Harvest Media (0.5% w/v HSA in Plasmalyte-A) and washed two to three times by centrifugation at about 900×g for about 10 minutes at about room temperature. The expanded NK cells can then be resuspended in NK Cell Harvest Media, or if desired, the pellet can be resuspended in a CD3-Depletion Buffer (such as CliniMACS® Buffer) in preparation for CD3-depletion. The NK Cell Thaw Media can also comprise 5% (w/v) HSA in 0.9% preservative-free normal saline or any other appropriate media known by a person of skill in the art.

As described previously, if T cell-depletion is not being performed at this time (after wash 2), the pellet comprising the NK cells can be resuspended and washed a third time in NK Cell Harvest Media by centrifugation at about 900×g for about 10 minutes at about room temperature. Following centrifugation (wash 3), the NK Cell Harvest Media can be removed or aspirated from the tube and the pellet comprising the NK cells can be resuspended in about 25 mL to about 100 mL of NK Cell Harvest Media.

Additionally or alternatively, other NK cell activating agents (i.e., stimulating agents) and stimulatory may be used for NK cell expansion. Examples of NK cell activating or stimulating agents and stimulatory peptides include, but are not limited to IL-21, 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, peptides, aptamers, TGFβ, and/or other homing inducing signaling molecules. Examples of cytokines include, but are not limited to, IL-2, IL-12, IL-21, and/or IL-18. Examples of aptamers include, but are not limited to IL-21, 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, TGFβ, and/or other homing inducing signaling molecules. Examples of adhesion molecules include, but are not limited to LFA-1, MICA, and/or BCM/SLAMF2. NK cell activating or stimulating agents can comprise individual molecules and/or be derived from cells. NK cell activating or stimulating agents may be added directly to the culture during NK cell expansion. NK cell activating or stimulating agents may also be delivered to the NK cells during expansion by a delivery vehicle. The delivery vehicle may comprise cells, including, but not limited to FCs. The delivery vehicle may comprise microparticles or nanoparticles, comprised of various materials. In addition, the delivery vehicle may comprise natural and/or artificial (e.g., synthetic) lipid bilayers or membranes. These NK cell effector agents can be soluble presented in solution or present as membrane bound agent on the surface of PM particles, EX, or FCs. The PM particles, EXs, and/or FCs can be engineered to express membrane forms of the NK cell activating agents and stimulatory peptides. Alternatively, the NK cell activating agents and stimulatory peptides can be chemically conjugated to the surface of the PM particle, EX, of FC. For example, a PM particle, FC, or EX prepared from FC expressing membrane bound IL-21. Thus, in one aspect, disclosed herein are isolated universal donor NK cells or cell lines wherein the universal donor NK cell or cell line is activated and/or expanded by incubating the universal donor NK cells in vitro in the presence of one or more activating agents, stimulatory peptides, cytokines, and/or adhesion molecules including, but not limited to 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, and/or TGFβ (for example, IL-21). In one aspect, the IL-21 used in the in vitro activation comprises soluble IL-21, FC21 s (also referred to herein as FCs), PM21s, or EX21s. It is understood and herein contemplated that the membrane bound IL-21 expressing FCs, PMs, and EXs can further comprise additional one or more activating agents, stimulatory peptides, cytokines, and/or adhesion molecules including, but not limited to 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, and/or TGFβ (for example, PMs, EXs, or FCs expressing 41BBL and membrane bound IL-21).

As noted above, the additional activation and/or expansion of the universal donor NK cells increases the efficacy of the cell when administered to a recipient. Thus, in one aspect, disclosed herein are methods for preparing a population of universal donor NK cells for therapeutic administration to a recipient subject in need thereof, the method comprising: (a) obtaining an initial population of NK cells from a NK cell donor, wherein the NK cell donor has a genotype indicating the presence of (i) at least two of variably inherited activating KIRs 2DS1/2, 2DS3/5, 3DS1, and/or 2DS4; and (ii) at least one, two, or all three HLA alleles comprising of C1, C2, and Bw4 alleles; and (b) exposing the initial population of NK cells to one or more activating agents, stimulatory peptides, cytokines, aptamers, and/or adhesion molecules including, but not limited to IL-21, 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, and/or TGFβ (for example, IL-21) in vitro for a time and under conditions sufficient to expand the initial population of NK cells. It is understood and herein contemplated that the exposure to the one or more activating agents can occur for a time and under conditions to achieve at least one population doubling.

In one aspect, the isolated universal donor NK cell or cell line or population of NK cells is characterized by increased ability to produce and secrete anti-tumor cytokines of IFNγ or TNFα. In one aspect, the expanded population of NK cells is characterized by increased expression of NKG2D, increased expression of CD16, increased expression of NKp46, and/or increased KIR expression.

Release Criteria and Final NK Cell Compositions

Final NK cells can comprise expanded NK cells that meet (i.e., pass) final release criteria. Following NK cell expansion and T cell-depletion, expanded NK cells that meet final release criteria (Final NK cells) can be diluted to a predetermined, final concentration and either immediately administered to a recipient subject or cryopreserved and stored for future administration.

In one aspect, final release criteria includes but is not limited to the following: a percentage of CD3+ cells (T cells and NK T cells) of less than or equal to about 5%, or about 1%, or about 0.33%; greater than about 90% CD3-CD56+ cells (NK cells); and less than about 1% of CD56-CD32+CD137L+ cells (K562 FCs). In another aspect of the present disclosure, the percentage may be calculated based on the total number of CD45+ cells (WBCs). Percentages of T cells (CD3+CD56− cells) and NK T cells (CD3+CD56+ cells), individually, may also be evaluated. Final release criteria can also include tests for contamination, for example, testing for mycoplasma contamination.

The Final NK cells can be diluted in an NK cell Cryopreservation Media to a predetermined concentration, cryopreserved, and stored within cryobags or cryovials until administration. Generally, NK cell Cryopreservation Media comprises a protein source (e.g., HSA or human AB serum), a cryoprotectant (e.g., DMSO), and a base media (e.g., Plasmalyte-A or other infusible-grade fluid- or electrolyte-replacement or buffer solution). The NK cell Cryopreservation Media may also comprise, STEM-CELLBANKER™ (Amsbio), which reportedly contains 5-10% DMSO and ≤10% inorganic salts and has been optimized for cryopreservation directly at about −80° C., without the need for a controlled rate freezer. Preferably, the NK cell Cryopreservation Media comprises, 10% DMSO and 12.5% (w/v) HSA in Plasmalyte-A and the Final NK cells are cryopreserved and stored at a temperature below about −150° C., in liquid or vapor phase nitrogen. Preferably, a freezer program can be utilized and a freeze curve may be generated and analyzed to verify a controlled freeze was successfully performed.

In one aspect, disclosed herein are Final NK cell compositions and methods of making the Final NK cell compositions. NK cell compositions comprise Final NK cells diluted in NK cell Cryopreservation Media to a predetermined, final concentration. The final concentrations of the Final NK cell compositions can include, but are not limited to the following: about 0.5×10⁵ cells/mL, about 0.1×10⁶ cells/mL, about 0.5×10⁶ cells/mL, about 1×10⁶ cells/mL, about 2×10⁶ cells/mL, about 3×10⁶ cells/mL, about 4×10⁶ cells/mL, about 0.5×10⁷ cells/mL, about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 10×10⁷ cells/mL, about 11×10⁷ cells/mL, about 12×10⁷ cells/mL, about 13×10⁷ cells/mL, about 14×10⁷ cells/mL, about 15×10⁷ cells/mL, about 16×10⁷ cells/mL, about 17×10⁷ cells/mL, about 18×10⁷ cells/mL, about 19×10⁷ cells/mL, about 20×10⁷ cells/mL.

Preferably, final concentrations of the Final NK cell compositions includes more than four different concentrations. Generally, the total volume of the individual, Final NK cell compositions is about 15 mL to about 30 mL, preferably about 20 mL to about 25 mL, when stored in cryobags or an equivalent sized cryo-storage container. The total volume of individual, Final NK cell compositions is about 0.1 mL to about 5 mL, preferably about 0.5 mL to about 1.8 mL, when stored in cryovials, or an equivalent sized cryo-storage vial or tube.

Also disclosed herein are methods of cryopreserving or storing the Final NK cell compositions. The Final NK cells compositions can be cryopreserved and stored at a temperature below about −150° C., in liquid or vapor phase nitrogen, or any other method cryopreservation method known to a person of skill in the art. Preferably, a freezer program can be utilized and a freeze curve may be generated and analyzed to verify a controlled freeze was successfully performed. Prior to administration to a recipient subject or patient, Final NK cell compositions are thawed, for example in water bath or bead bath, in a temperature of about 37° C.

Final NK cell compositions can be identified by the batch number of its Final NK cells. The batch number can provide the following information including, but not limited to, the donor, specimen collection and processing, the date the MNCs entered or began the NK expansion process, and details regarding the NK expansion, such as any viability and immunophenotypic analysis that was performed on the NK Expanded cells as they proceeded through the expansion process. Each Final NK cell composition batch may also have a lot numbers. The lot number of a particular batch, identifies the concentration of Final NK cells within the Final NK cell composition as well as the number of Endotoxin units, and details of the freezing program. A single batch may only have a single lot or a single batch may have more than one lot. In addition, single lot may only comprise a Final NK cell composition that is stored within a single cryo-storage container (e.g., one cryobag or one cryovial). Additionally or alternatively, a single lot may also comprise a Final NK cell composition that is stored within multiple cryo-storage containers (e.g., cryobags and/or cryovials).

All lots of Final NK cell compositions can be tested for the number of Endotoxin units. Analysis of Endotoxin units can be performed by any method known to a person of ordinary skill in the art.

Clinical Decision Support System and Algorithms

In one embodiment, disclosed herein are methods and algorithms that can be used to analyze recipient subjects and determine safe and efficient dosing of Final NK cell compositions in a patient or recipient subject for the treatment or prevention of cancer and/or disease.

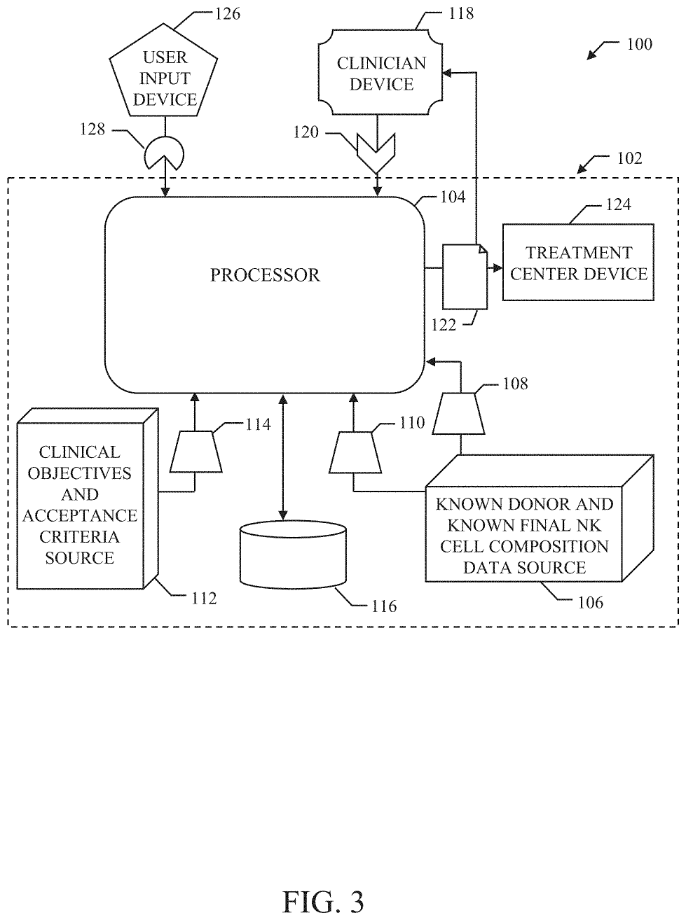

FIG. 3 is a diagram of a clinical decision support system 100 according to an example embodiment of the present disclosure. The example system 100 includes a clinical management server 102, which is configured to process/update the procedure 200 disclosed herein and provide treatment decisions using the algorithms. The clinical management server 102 includes a processor 104 configured to process the clinical decision algorithms disclosed herein. While the clinical management server 102 shows only one processor 104, in other embodiments, the processor 104 may be separated into one or more processors capable of performing different tasks.

It should be understood that the operations described in connection with the processor 104 may be implemented using one or more computer programs or components. The programs of the components may be provided as a series of computer instructions on any computer-readable medium, including random access memory (“RAM”), read only memory (“ROM”), flash memory, magnetic or optical disks, optical memory, or other storage media. The instructions may be configured to be executed by a processor of the clinical management server 102, which when executing the series of computer instructions performs or facilitates the performance of all or part of the disclosed methods and procedures.

As shown in FIG. 3, the processor 104 is communicatively coupled to a known donor and Final NK cell composition data source 106, which may include a memory device storing known donor data 108 and may also include a memory device storing Final NK cell composition data 110. The processor 104 is also communicatively coupled to a clinical objectives and acceptance criteria source 112, which may include a memory device storing clinical objectives and acceptance criteria source 114, for the clinical decision algorithms. The processor 104 may store the clinical decision algorithms to a memory device 116.

In some embodiments, the processor 104 hosts a website or other Internet accessible interface, such as an application programmable interface (“API”) that enables clinician devices 118 to submit patient or recipient subject data and receive treatment administration information. In another embodiment, the clinician device 118 may be included as part of a service portal, a HIPAA compliant database, and electronic medical records databases (not shown).

The processor 104 may receive recipient subject or patient data 120. In one embodiment, after processor 104 applies the data to the clinical decision algorithms and treatment decisions are made, the processor 104 creates a treatment plan and a dosing schedule 122. The processor 104 may display the information from the treatment plan and a dosing schedule 122 in a user interface, such as a webpage or interface with the clinician device 118. The processor 104 may display (in a user interface) or send the information from the treatment plan and a dosing schedule 122 to the treatment center device 124. In one aspect, similar to the clinician device 118, the processor 104 hosts a website or other Internet accessible interface that enables the treatment center devices 124 to receive the treatment plan and dosing schedule 122. In one embodiment, the treatment center device 124 may be physically located where the Final NK cell compositions are processed and stored, and/or may also be where the treatment center to administer the Final NK cell compositions is located.

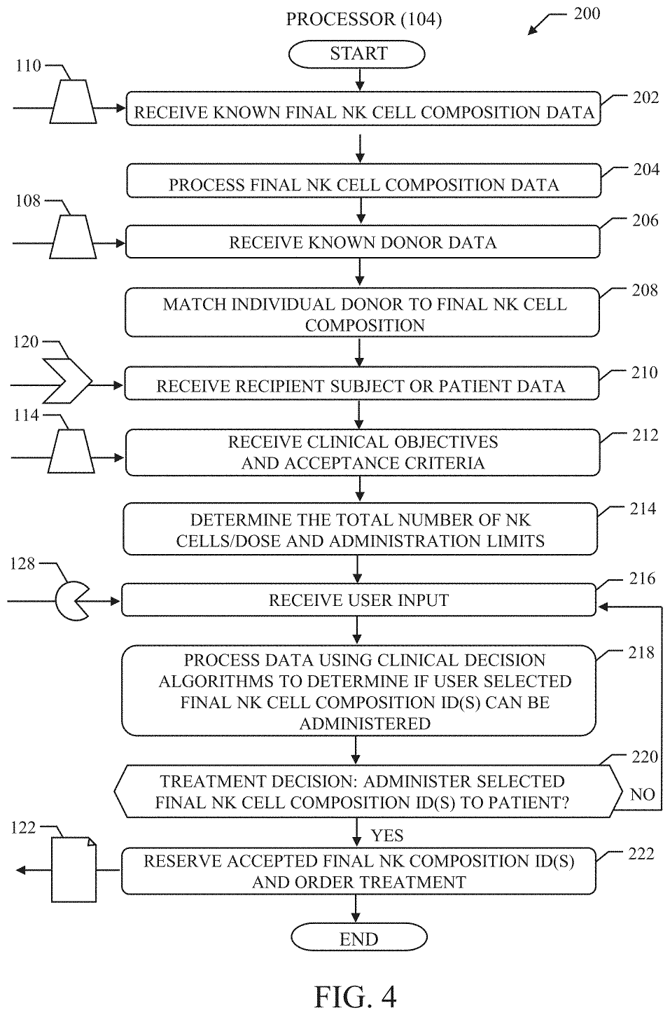

FIG. 4 is a flow diagram of an example procedure 200 for analyzing a recipient subject's data, known donor data, and known Final NK cell composition data via the clinical decision algorithms disclosed herein, according to an example embodiment of the present disclosure. Although the procedure 200 is described with reference to the flow diagram illustrated in FIG. 4, it should be appreciated that many other methods of performing the steps associated with the procedure 200 may be used. For example, the order of many of the blocks may be changed, certain blocks may be combined with other blocks, and many of the blocks described may be optional. In an embodiment, the number of blocks may be changed based data preprocessing and filtering and/or the types of clinical decision algorithms being developed. The actions described in the procedure 200 are specified by one or more instructions that are stored in a memory device 116, and may be performed among multiple devices including, for example the processor 104.

The example procedure 200 begins when the processor 104 receives known Final NK cell composition data 110 from, for example, a known donor and Final NK cell composition data source 106 (block 202). The known Final NK cell composition data 110 may include, the following information, which is tied to the unique ID of an individually, cryo-stored Final NK cell composition: the corresponding donor ID, batch number, lot number, the date the MNCs were T cell-depleted, the date the MNCs entered the NK cell expansion process, the date the NK cells were harvested, any and all results from cell viability immunophenotypic analysis performed before or after of the aforementioned procedures.

In addition, the known Final NK cell composition data 110 may include, the following information about the Final NK cell composition, the type of cryo-container the Final NK cell composition is stored in (e.g., cryobag or cryovial), the total volume of NK cell Cryopreservation Media, the total number of WBCs (CD45+ cells), the total number of NK cells/mL (CD3-CD56+ cells), percentages of NK cells, combined T cells and NK T cells (CD3+ cells), FCs (CD56-CD32+CD137L+ cells), Endotoxin U/mL, and the storage location of the Final NK cell composition.

In another embodiment, in an example procedure 200, the processor 104 performs calculations from known Final NK cell composition data 110, to determine the following for Final NK cell composition IDs: the number of Endotoxin Units, total number of NK cells (CD3−CD56+ cells), total number of CD3+ cells (i.e., both T cells and NK T cells), total number of T cells (CD3+CD56− cells), and total number of NK T cells (CD3+CD56+ cells) (block 204).

The processor 104 may also receive known donor data 108 from, for example, a known donor and Final NK cell composition data source 106 (block 206). The known donor data 108 may include the date the donor's specimen was collected, the type of specimen collected (i.e., whole blood or plasma), the method of collection used (i.e., phlebotomy or apheresis), and the donor's HLA-genotype, including but not limited to, the presence or absence of various alleles for HLA class I and/or HLA class II. In one embodiment, the known donor data 108 may contain health history including, but not limited to, infectious disease diagnosis, immune disorders, blood disorders, current medications, etc. The known donor and Final NK cell composition data source 106 may include one or more electronic medical records (“EMR”) databases that are located at clinics or hospitals and store electronic information concerning the donors.

The processor 104 can link the donor to a particular Final NK cell composition that comprises that particular donor's NK cells by matching the unique donor ID (known donor data 108) to the donor ID information that is associated with a Final NK cell composition ID (known Final NK cell composition data 110) (block 208).

In one embodiment, disclosed herein the processor 104 may receive recipient subject or patient data 120 from, for example, a clinician device 118 (block 210). The recipient subject or patient data 120 may include the demographic data such as age, gender, race, and other personally identifiable data (PII). The patient recipient subject or patient data 120 may also include physiological data, such as weight (kg), body surface area (m²), blood pressure, temperature, hemoglobin levels, and protected health information (PHI) here. The patient recipient subject or patient data 120 may include instructions from the physician or healthcare provider regarding the desired dose (NK cells/kg or NK cells/m²), the number of doses, and/or the dosing schedule. Additionally, the recipient subject or patient data 120 may include past or current health history such as current conditions, diseases, disorders, current medications, the recipient subject's HLA-specific antibody profile, including but not limited to, the presence and quantity (MFI) of HLA class I- and class II-specific antibodies.

The processor 104 may also receive clinical objectives and acceptance criteria 114 from, for example, a clinical objectives and acceptance criteria source 112 (block 212). The clinical objectives and acceptance criteria 114, may include the maximum CD3+ cell dose (cells/kg), the maximum amount of Endotoxin Units (EU)/10⁸ cells or EU/kg, the maximum quantity of HLA class I-specific antibodies and/or HLA class II-specific antibodies that excludes a recipient subject from receiving NK cells from a HLA-positive donor, and the degree the NK cell dose may safely deviate from the calculated dose (i.e., ±20% of the total number of NK cells/dose). In addition, the clinical objectives and acceptance criteria 114, may include and the maximum cumulative CD3+ cell dose across a two week period of time.

In another embodiment, using the received clinical objectives and acceptance criteria 114 and the recipient subject or patient data 120, such as the patient's weight (kg) and desired dose (NK cells/kg), the processor 104 determines the total number of NK cells/dose, the limit of CD3+ cells/dose, as well as the upper and lower limits of the NK cell dose, i.e., the acceptable administration range of total NK cells/dose (block 214). It is understood, and herein contemplated, that equivalent calculations for other measurement units, such as m² for body surface area, can also be performed by the processor 104 under example procedure 200.

The example processor 104 may also receive input from the user 128 via an input device 126. The user voluntarily selects Final NK cell composition ID(s) which, in this example procedure 200, are visible displayed in a user interface (block 216). After receiving the input, the processor 104 uses the clinical decision algorithms to make a treatment decision regarding whether the selected Final NK cell composition ID(s) can be administered to the recipient subject or patient.

Using the clinical decision algorithms, the processor 104 processes the known donor data 108, and/or known Final NK cell composition data 110, and/or the clinical objectives and acceptance criteria 114, and/or the recipient subject or patient data 120 to make a treatment decision, i.e., to determine if the Final NK cell composition ID(s), selected by the user input 128, can be administered to the recipient subject or patient (block 218). For example, the clinical decision algorithms will determine whether the recipient subject's HLA antibody profile, including the presence and or quantity of HLA class I- and class II-specific antibodies, is compatible with the Final NK cell composition ID's donor's HLA type. In one embodiment, the processor 104 can use the clinical decision algorithms to determine if the Final NK cell composition ID(s), selected by the user input 128, comprise a total NK cells that falls within the acceptable administration dose range. In another embodiment, the processor 104 can use the clinical decision algorithms to determine if the Final NK cell composition ID(s), selected by the user input 128, exceed the maximum of CD3+ cells, the maximum Endotoxin U/dose. In addition, in this example procedure 200, the processor 104 does not allow a user to select a Final NK cell composition ID that has been previously selected and reserved for a patient or recipient subject.

If the clinical decision algorithms determined that selected Final NK cell composition ID(s) were not acceptable, such as over certain maximum limits, out of a specific range, potentially cross-reactive with the recipient subject, etc., then the treatment decision is NO (block 220). The user will have to deselect the selected Final NK cell composition ID(s) to deselect one or more Final NK cell composition ID's and begin selecting other Final NK cell composition ID(s) (block 216).

If the clinical decision algorithms determined that selected Final NK cell composition ID(s) were acceptable, then the treatment decision is YES and the processor 104 reserves the selected Final NK cell composition ID(s) and creates a treatment plan and dosing schedule 122 in a user interface, such as a webpage or an interface of the clinician device 118 and/or the treatment center device 124 (block 222). The treatment plan and dosing schedule 122 includes, but is not limited to, patient or recipient subject identifying information, the dosing information, the Final NK cell composition ID(s) and their freezer storage location. The example procedure 200 ends until, a treatment decision is needed for another patient or when the same patient requires another dose.

The system allows for future donor-recipient non-match scenarios to be included. In addition, the system also contemplates included pre-treatment instructions if a pre-treatment may prevent a donor-recipient non-match scenario.

Methods of Treating Disease or Cancer

In one embodiment, disclosed herein is a method of treating a recipient subject or patient, comprising: (a) testing the recipient subject for HLA-specific antibodies; (b) excluding potentially cross-reactive Final NK cell compositions; (c) measuring the recipient subject's body surface area or weight; and (d) calculating the Final NK cell dose level or the total number of Final NK cells to be administered at one time (i.e., one dose). In one embodiment the recipient subject is a mammal. The recipient subject is preferably a human.

Methods of testing the recipient subject or patient for HLA-specific antibodies can include any method known to a person of ordinary skill in the art. The testing of recipient subjects for HLA-specific antibodies prior to dosing or therapeutic administration of Final NK cell compositions can help identify the appropriate or desired Final NK cell composition to be administered. Final NK cell compositions from donors with potentially cross-reactive HLA types can be excluded when selecting the Final NK cell composition to be administered to the recipient subject. Importantly, screening of patients for HLA-specific antibodies and excluding potentially cross-reactive Final NK cell compositions can minimize immediate immune-mediated rejection of administered (or infused) Final NK cell compositions, and may also reduce the likelihood of immune-mediated infusion reactions.

In addition, a person of ordinary skill in the art would understand that there are additional limits to be considered when administering compositions, such as a maximum Endotoxin Units/dose, a maximum dose of DMSO, a maximum dose of CD3+ cells/dose, etc. It is understood and herein contemplated that the method of treatment may include calculating the aforementioned excipients, toxins, cells, etc., prior to administering the Final NK cell composition.

It is understood and herein contemplated that body surface area, weight, and/or any other acceptable measurements known to a person of skill in the art, may be used to calculate or determine the dose or the appropriate total number of Final NK cells (i.e., the selection of the Final NK cell composition) to be administered, at one time, to a recipient subject or patient.

Final NK cell compositions with different final concentrations of Final NK cells allows for a physician or health care professional to select and administer a composition comprising a precise dose for that particular recipient subject or patient. This not only reduces Final NK cell waste of any remaining composition (especially for small doses), but serves as safety measure to avoid possible overdosing.

In one aspect, disclosed herein are methods of treating, preventing, inhibiting, and/or reducing a cancer, metastasis, or a disease in a subject comprising administering to the recipient subject or patient any of the isolated or engineered universal donor NK cell or cell line disclosed herein or any universal donor NK cell or cell line or engineered universal donor NK cell or cell line that is selected by or screened by the method or prepared by any of the methods disclosed herein. For example, in one aspect, disclosed herein are methods of treating a cancer or a disease in a subject comprising (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1; (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; and (c) selecting the candidate NK cells as a universal donor NK cell for the therapeutic administration when (i) the HLA genotype indicates the presence of at least two HLA alleles HLA C1, C2, and Bw4; and (ii) the KIR genotype indicates the presence of at least three activating KIRs 2DS1/2, 2DS3/5, 3DS1, and/or 2DS4.

In another non-limiting aspect disclosed herein, are methods treating a cancer or an infectious disease, wherein the selected universal donor NK cells are histologically optimized with at least 50%-85% of recipient subjects. In one aspect the methods of treating a cancer or an infectious disease of any preceding aspect, further comprising obtaining or having obtained the CMV seropositivity of the candidate NK cells; and wherein the NK candidate NK cells are further selected when the NK cell donor is seropositive for CMV or the NK cells from the NK cell donor have high NKG2C expression compared to a reference level of NKG2C expression.

It is understood and herein contemplated that activating and/or expanding the universal donor NK cells prior to therapeutic administration to a recipient subject can help overcome many hurdles associated with cytokine toxicity. In one aspect, the methods treating a cancer or an infectious disease of any preceding aspect, further comprising incubating the selected universal donor NK cells in vitro in the presence of one or more NK cell effector agents (i.e., stimulatory peptides, cytokines, and/or adhesion molecules), for example IL-21. Examples of NK cell activating agents and stimulatory peptides include, but are not limited to IL-21, 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, TGFβ, and/or other homing inducing signaling molecules. Examples of cytokines include, but are not limited to, IL-2, IL-12, IL-21, and IL-18. Examples of adhesion molecules include, but are not limited to LFA-1, MICA, and BCM/SLAMF2. These NK cell effector agents can be soluble presented in solution or present as membrane bound agent on the surface of PM particles, EX, or FCs. The PM particles, EXs, and/or FCs can be engineered to express membrane forms of the NK cell activating agents and stimulatory peptides. Alternatively, the NK cell activating agents and stimulatory peptides can be chemically conjugated to the surface of the PM particle, EX, or FC. For example, a PM particle, FC, or EX prepared from FCs expressing membrane bound IL-21. It is understood and herein contemplated that the membrane bound IL-21 expressing FCs, PMs, and EXs can further comprise additional one or more activating agents, stimulatory peptides, cytokines, and/or adhesion molecules including, but not limited to 41BBL, IL-2, IL-12, IL-15, IL-18, IL-7, ULBP, MICA, LFA-1, 2B4, BCM/SLAMF2, CCR7, OX40L, NKG2D agonists, Delta-1, Notch ligands, NKp46 agonists, NKp44 agonists, NKp30 agonists, other NCR agonists, CD16 agonists, and/or TGFβ (for example, PMs, EXs, or FCs expressing 41BBL and membrane bound IL-21).

It is understood that the pathogen can be a virus. Thus in one embodiment the pathogen can be selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods wherein the pathogen is a bacterium. The pathogen can be selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acinetobacter baumannii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella petrii, Bordetella parapertussis, Bordetella ansorpii, other Bordetella species, Burkholderia mallei, Burkholderia pseudomallei, Burkholderia cepacia, Bacillus anthracia, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neisserria meningitidis, Neisserria gonorrhoeae, Pseudomonas aeruginosa, other Pseudomonas species, Haemophilus influenzae, Haemophilus ducreyi, other Hemophilus species, Clostridium tetani, other Clostridium species, Yersinia enterocolitica, and other Yersinia species, and Mycoplasma species.

Also disclosed are methods of treating an infectious disease wherein the pathogen is a fungus selected from the group of fungi consisting of Candida albicans, Cryptococcus neoformans, Histoplasma capsulatum, Aspergillus fumigatus, Coccidioides immitis, Paracoccidioides brasiliensis, Blastomyces dermatitidis, Pneumocystis carinii, Penicillium marneffei, and Alternaria alternata.

Also disclosed are methods of treating an infectious disease wherein the pathogen is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, and Trichinella nativa.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, thyroid cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, stomach cancer, prostatic cancer, or pancreatic cancer.

It is also contemplated herein that the disclosed methods of treating, preventing, inhibiting, or reducing a cancer or metastasis in a subject can further comprise the administration of any anti-cancer agent that would further aid in the reduction, inhibition, treatment, and/or elimination of the cancer or metastasis (such as, for example, gemcitabine). Anti-cancer agents that can be used in the disclosed bioresponsive hydrogels or as an additional therapeutic agent in addition to the disclosed pharmaceutical compositions, engineered particles, and/or bioresponsive hydrogels (including bioresponsive hydrogels that have an engineered particle encapsulated therein) for the methods of reducing, inhibiting, treating, and/or eliminating a cancer or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant Human Papillomavirus (HPV) Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanli sib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Ti sagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant HPV Bivalent Vaccine, Recombinant HPV Nonavalent Vaccine, Recombinant HPV Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Ti sagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with coloroimetric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Generally, fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1™; Cy3.5™; Cy3™; Cy5.1™; Cy5.5™; Cy5™; Cy7™; Cyan GFP; cyclic AMP (cAMP) Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′ DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (Di1C18(5)); DIDS; Dihydorhodamine 123 (DHR); Dil (Di1C18(3)); I Dinitrophenol; DiO (DiOC18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type (WT) non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red□; Texas Red-X□ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, and fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avidin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electrophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulfate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulfide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log₁₀MW for known samples, and read off the logMr of the sample after measuring distance migrated on the same gel.

In two-dimensional (2D) electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromogenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I. Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. RIPA is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (MA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. MA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, a-glycerophosphate dehydrogenase, triose phosphate isomerase, glucose-6-phosphate dehydrogenase, glucoamylase and acetylcholinesterase.

Variations of ELISA techniques are known to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISAs, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immune complexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immune complex (antigen/antibody) formation. Detection of the immune complex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, a-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immune complex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° C. to 30° C., or can be incubated overnight at 30° C., about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immune complexes between the test sample and the originally bound material, and subsequent washing, the occurrence of even minute amounts of immune complexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immune complex with a labeled antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include color coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per cm², at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.), and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the program range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulfide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, Conn.).

As a 2D display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### EXAMPLES

### Example 1

Three clones of FCs from the MCB were cultured. FCs were cultured and manually counted every two to four days. Cultures were split 1:10 when they exceeded 1×10⁶ cells/mL. Cultures were continued until at least 100×10⁶ cells were obtained. The three clones were pooled and centrifuged at 350×g for 5 minutes at room temperature. Cells were resuspended in RPMI Complete Media (with GlutaMAX and 10% FBS), targeting 10×10⁷ cells/mL. Cells were manually counted again and re-diluted to 6.67×10⁷ cells/mL. 2× Freeze Media was prepared from RPMI Complete Media by adding 20% DMSO. Four different FC cryo-concentrations were prepared according to Table 3, in 1.5 ml cryovials. The FCs were cryopreserved at below −150° C., in a LN₂ vapor phase freezer for a minimum of 24 hours. A freezer program was used and successful controlled freeze was verified by a freeze curve.

TABLE 3
Preparation of Feeder Cell (FC) Cryo-Concentrations

| Cryo-Concentration | Volume | Volume of RPMI | | Volume of 2X |
|---|---|---|---|---|
| (FCs/1.5 mL Cryovial) | of FCs | Complete Media | | Freeze Media |
| 5 × 10⁶ | 0.75 mL of | 0 | mL | 0.75 mL |
| | 1:10 dilution | | | |
| 1 × 10⁷ | 0.15 mL | 0.60 | mL | 0.75 mL |
| 2 × 10⁷ | 0.3 mL | 0.45 | mL | 0.75 mL |
| 5 × 10⁷ | 0.75 mL | 0 | mL | 0.75 mL |

Following cryopreservation, individual cryo-concentrations were thawed and diluted to 10 mL with RPMI Complete Media (with GlutaMAX and 10% FBS) and centrifuged at 350×g for 10 minutes at room temperature. Pellets were resuspended in 5 mL RPMI Complete Media (with GlutaMAX and 10% FBS) and the FCs were assessed using a manual cell count and trypan blue assay. Percent cell viability (i.e., the number of live cells/number of total cells (live+dead)×100), percent cell recovery (i.e., the number of live cells after/number of live cells before×100), and the alive cell number was assessed following a minimum of 24 hours of cryopreservation.

TABLE 4
Viability and Recovery of Feeder Cells
(FCs) in Different Cryo-Concentrations

| Cryo-Concentration | Viability | Recovery | Alive Cell |
|---|---|---|---|
| (Cells/1.5 mL Cryovial) | (%) | (%) | Number |
| 5 × 10⁶ | 86.85 | 77.63 | 0.39 × 10⁷ |
| 1 × 10⁷ | 91.73 | 85.96 | 0.86 × 10⁷ |
| 2 × 10⁷ | 89.30 | 88.75 | 1.78 × 10⁷ |
| 5 × 10⁷ | 91.07 | 84.13 | 4.21 × 10⁷ |

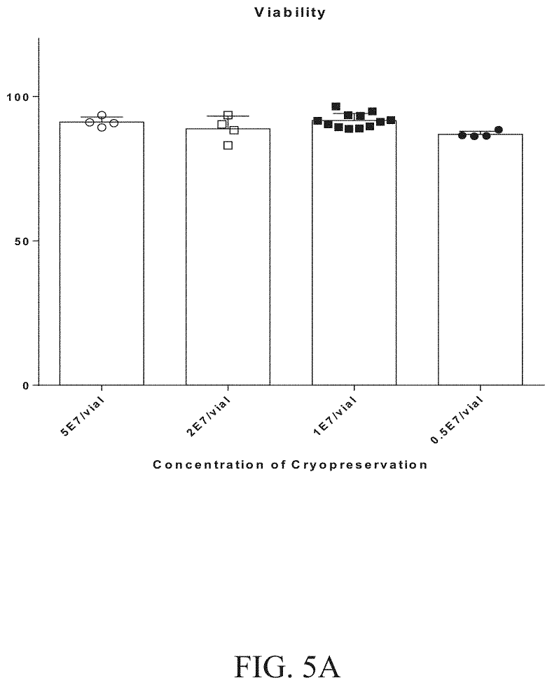

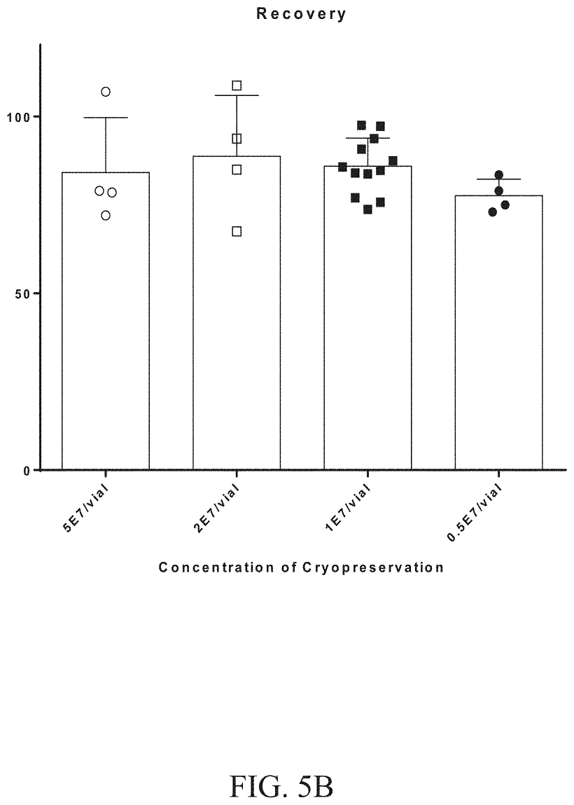

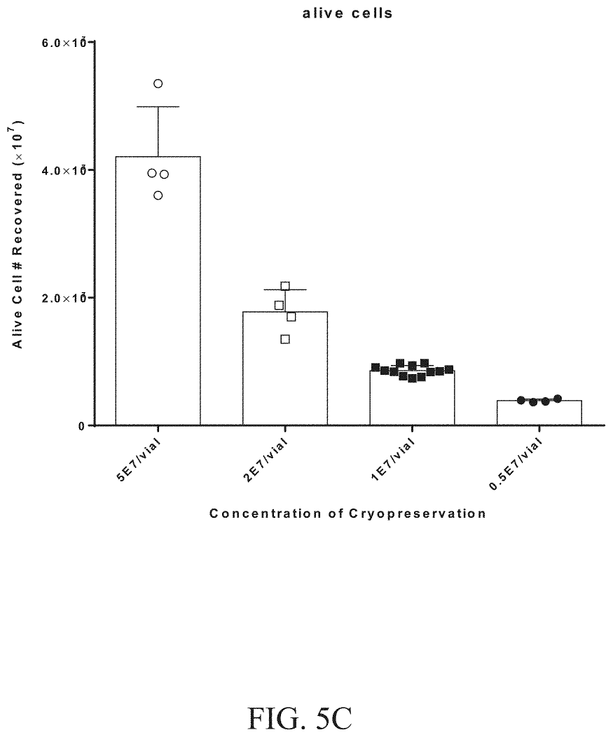

For all the cryo-concentrations tested, both percent viability (FIG. 5A) and percent recovery (FIG. 5B) were higher than 75%. In addition, the number of alive FCs after thawing (FIG. 5C) was consistent with the cell number before cryopreservation for all four cryo-concentration groups. Thus, no significant differences were observed among the different cryo-concentration groups of FCs (Table 4).

### Example 2

FCs, from the MCB, were cultured and manually counted on the first day. Cultures were maintained for a sufficient number of days, then the FCs were harvested, transferred to sterile conical tubes and centrifuged at 500×g for 10 minutes at room temperature. The supernatant was removed and the pellet was resuspended in RPMI Complete Media (with GlutaMAX and 10% FBS), to approximately 10×10⁷ cells/mL based on the day-1 count. A manual cell count was then performed, and the FCs were further diluted with RPMI Complete Media (with GlutaMAX and 10% FBS) to achieve 8×10⁷ cells/mL.

FCs were irradiated with gamma radiation at 100 Gy. Following irradiation, the IFCs were diluted to two different concentrations according to Table 5, using RPMI Complete Media (with GlutaMAX and 10% FBS) and 2× Freeze Media, in individual 25 mL cryobags. 2× Freeze Media was previously prepared from RPMI Complete Media (with GlutaMAX and 10% FBS) and 20% DMSO. The IFCs were cryopreserved at below −150° C., in a LN₂ vapor phase freezer for a minimum of 24 hours. A freezer program was used and successful controlled freeze was verified by a freeze curve.

TABLE 5
Preparation of Irradiated Feeder Cell
(IFC) Cryo-Concentrations in Cryobags

| | | | Number |
|---|---|---|---|
| IFC Cryo- | Cells/25 mL | | of 25 mL |
| Concentration | Cryobag | Cells/mL | Cryobags |
| Low | 0.25 × 10⁹ | 1 × 10⁷ | 8 |
| Medium | 0.5 × 10⁹ | 2 × 10⁷ | 7 |

Following cryopreservation, individual cryo-concentrations were thawed and diluted to 10 mL with RPMI Complete Media (with GlutaMAX and 10% FBS), centrifuged at 350×g for 10 minutes at room temperature, then the pellets were resuspended and manually counted. Percent cell viability, percent cell recovery, and the alive cell number, from individual cryobags for both the Low and Medium cryo-concentrations was assessed using the trypan blue viability assay. (Table 6).

TABLE 6
Viability and Recovery of Irradiated Feeder Cells (IFCs)
in Low and Medium Cryo-Concentrations in Cryobags

| | Number of | | | | Theoretical | |
|---|---|---|---|---|---|---|
| | Viable | | | | Viable | |
| Cryo- | Cells/mL | | Viability | | Cell Recovery | |
| Concentration | Mean | Std Dev | Mean | Std Dev | Mean | Std Dev |
| Low | 9.56 × 10⁶ | 1.71 × 10⁶ | 60% | 6% | 96% | 17% |
| Medium | 1.53 × 10⁶ | 1.71 × 10⁶ | 46% | 5% | 80% | 28% |

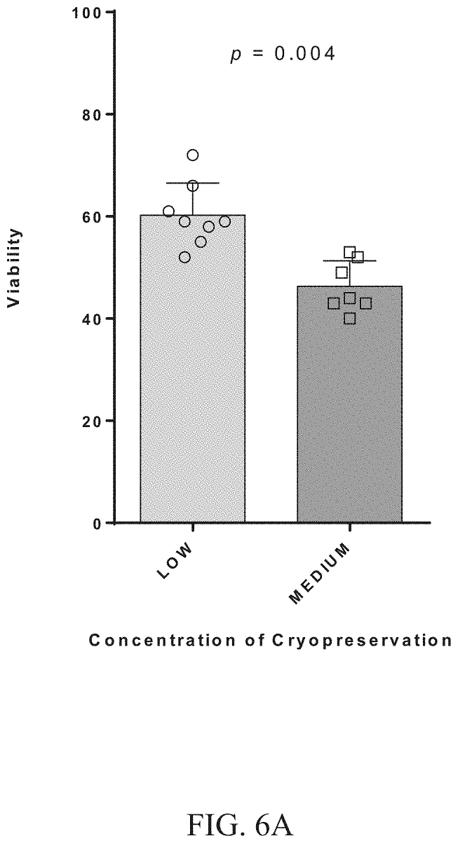

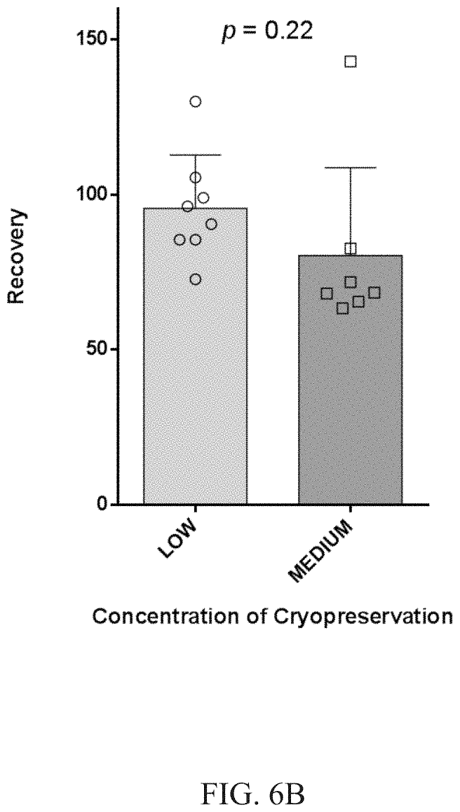

Both viability (46% vs. 60%; FIG. 6A) and theoretical viable cell recovery (80% vs. 96%; FIG. 6B) appear to be lower for the medium cryo-concentration cryobags compared to the low cryo-concentration cryobags. In unpaired t-tests, the difference in mean viability (p<0.004), but not viable cell recovery (p=0.22), between the medium cryo-concentration group and the low cryo-concentration group was statistically significant (Table 6).

### Example 3

Fresh whole blood specimens were collected from normal donors. A total of eight, 50 mL specimens were immediately processed for MNC isolation and enrichment using SepMate™ conical tubes and low speed centrifugation. Next the MNC samples underwent T cell-depletion (i.e., CD3-depletion) using the Miltenyi Biotec SuperMACS® Magnet System with XS column. T cell-depletion was performed immediately for six of the MNC samples, while two MNC samples underwent delayed T cell-depletion; one was held at room temperature for 24 hours and the second was held at room temperature for 48 hours, prior to T cell-depletion. Among the six MNC samples that underwent immediate T cell-depletion, two samples underwent T cell-depletion utilizing an expired CD3 reagent, Miltenyi Biotec CliniMACS® CD3 MicroBeads. Specifically, one of these samples used CliniMACS® CD3 MicroBeads that were 21 days past-expiration date and had been open for a total of 42 days. The second sample (obtained from the same donor specimen) used CliniMACS® CD3 MicroBeads 29 days past-expiration and had been open for a total of 53 days. All other samples used non-expired CliniMACS® CD3 MicroBeads that had been open for less than one month. The BD PhaSeal™ system was used to allow multiple aliquots to be removed from the costly CliniMACS® CD3 MicroBead vials over time, while preventing contamination and reagent degradation and allowing multiple aliquots to be removed from expensive reagent vials that are manufactured for large MNC preparations such as an apheresis collection.

All samples underwent immunophenotypic analysis before CD3-depletion. After CD3-depletion, samples were diluted to 100 mL in CliniMACS® Buffer prior to viability and immunophenotypic analysis.

Following CD3-depletion, CD3-depleted MNCs were analyzed for percent cell viability. All eight CD3-depleted samples met the expected criteria for viability ≥70%, with mean of 99.2% (±0.3). This includes the samples that were held at room temperature for 24 or 48 hours prior to processing.

All eight CD3-depleted samples also met the expected criteria for percentage of T cells and NK T cells (CD3+ cells)<5%, with mean of 0.14% (±0.21%, median 0.05%) (Table 7). NK cell recovery from MNCs after CD3-depletion was 83% (mean±49%, median 85%).

TABLE 7
Immunophenotype and NK Cell Recovery
Following T Cell-Depletion

| | | | Log T Cell | |
|---|---|---|---|---|
| | T/NK T Cells | Fold NK Cell | and NK T Cell | NK Cell |
| | (CD3+ cells) | Enrichment | Depletion | Recovery |
| Median | 0.05% | 2.9 | 3.4 | 85% |
| Mean | 0.14% | 3.2 | 3.3 | 83% |
| Std Dev | 0.21% | 0.8 | 0.6 | 49% |

Statistical comparisons were made between the CD3-depleted samples that utilized expired CliniMACS® Buffer vials compared to the samples that utilized the non-expired CliniMACS® Buffer vials. The BD PhaSeal™ system was used for all CliniMACS® Buffer vials. No differences in results were seen between the CD3-depleted MNC samples that used expired CliniMACS® Buffer and the CD3-depleted MNC samples that used non-expired CliniMACS® Buffer (data not shown). The results confirm that expired CliniMACS® Buffer vials can be successfully used for CD3-depletion of MNCs and that the BD PhaSeal™ system can successfully prevent reagent degradation in open CliniMACS® Buffer vials.

Microbial testing was performed on the expired CliniMACS® Buffer vial open for 42 days, a non-expired vial open for 23 days, and a non-expired vial open for 95 days. The BD PhaSeal™ system was used on all CliniMACS® Buffer vials and vials were stored at 2-8° C. between runs. A positive control sample was also tested. The positive control consisted of a CliniMACS® Buffer vial that was 4 days past its expiration date and had been intentionally contaminated with a syringe tip prior to Bactec inoculation. All four samples of CliniMACS® Buffer were submitted for microbial cultures and no growth was reported for all samples except the positive control (data not shown). The results confirm that the BD PhaSeal™ system can successfully prevent microbial contamination in open CliniMACS® Buffer vials.

### Example 4

Fresh whole blood specimens were collected from two healthy donors on two different days in sodium heparin blood collection tubes and processed. The four MNC samples were CD3-depleted as previously described in Example 3. Following CD3-depletion, each of the four samples were split into two fractions prior to cryopreservation and storage. One fraction of each sample was diluted with Control In-Process Cryoprotectant Media, comprised of 10% DMSO, 6.25% (w/v) HSA, and 40% Plasmalyte-A, and the other fraction was diluted with Test In-Process Cryoprotectant Media, comprised of 10% DMSO and 40% FBS in 50% RPMI Complete Media with GlutaMAX. All sample fractions were diluted to a maximum concentration of 200×10⁶ cells/mL and stored in at least four 1.5 mL cryovials (each cryovial contained approximately 1 mL) for cryopreservation below −150° C., in a LN₂ vapor phase freezer, for a minimum of 3 days. A freezer program was used and successful controlled freeze was verified by a freeze curve.

Sample fractions were thawed in a 37° C. water bath and diluted 1:5 with Thaw Media, comprised of RFG-NK Expansion Media and 50 U/mL DNase with 1 mM MgCl₂ (RFG-NK Expansion Media is comprised of RPMI Complete Media with GlutaMAX and 10% FBS). Both fractions from Donor 1 were washed by centrifuging at 500×g for 10 minutes at room temperature with a 50% brake and resuspended in 10 mL of Thaw Media. However, it was observed that among the Donor 1 samples, the Control fractions did not resuspend well. Thus, the centrifugation speed was changed to 300×g for the washing of the remaining, Donor 2 samples. The slower centrifugation speed (300×g) resulted in improved resuspension for the Control fractions.

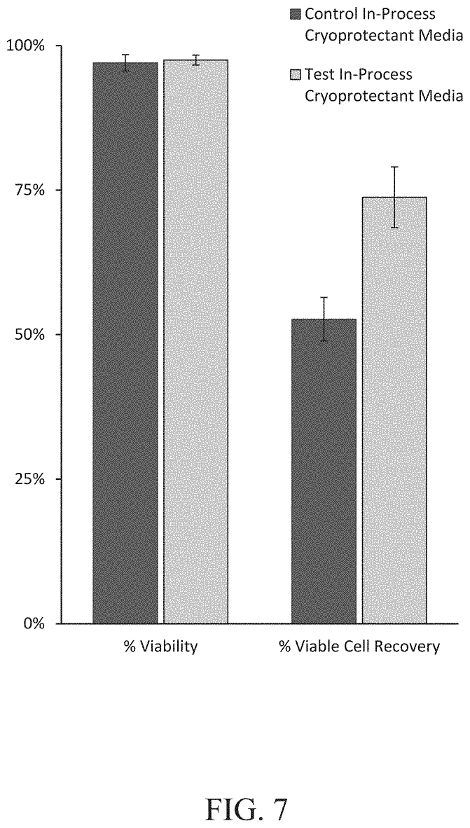

Manual cell counts were performed to assess percent cell viability and percent cell recovery. The results showed that CD3-depleted MNCs cryopreserved in the Test In-Process Cryoprotectant Media (10% DMSO and 40% FBS in 50% RPMI Complete Media with GlutaMAX) resulted in improved percent cell viability (98%±1%) and percent cell recovery (74%±5%), compared to CD3-depleted MNCs cryopreserved in Control In-Process Cryoprotectant Media (10% DMSO, 6.25% (w/v) HSA, and 40% Plasmalyte-A); percent cell viability (97%±1%) and percent cell recovery (53%±4%) (see Table 8; FIG. 7).

TABLE 8
Comparison of In-Process Cryoprotectant Media on CD3-Depleted MNCs
After Cryopreservation in 1.5 mL Cryovials, Thawing, And Washing

| | Cell Viability (%) | | Cell Recovery (%) | |
|---|---|---|---|---|
| | Control In-Process | Test In-Process | Control In-Process | Test In-Process |
| | Cryoprotectant | Cryoprotectant | Cryoprotectant | Cryoprotectant |
| Media | Media | Media | Media | Media |
| Mean | 97% | 98% | 53% | 74% |
| Std Dev | 1% | 1% | 4% | 5% |

In addition, based on the unexpected observations described above, a comparison of for samples washed at the two different centrifugation speeds (500×g vs. 300×g), within the Test and Control Cryo Media groups, revealed that the 300×g resulted in improved percent cell viability and percent cell recovery (data not shown).

### Example 5

The following study was performed retrospectively to compare clinical scale CD3-depleted MNCs cryopreserved in larger cryo-storage containers (i.e., cryobags) in the two different In-Process Cryoprotectant Media.

During pre-clinical process validation studies, fresh whole blood specimens were collected from five normal donors. The specimens were immediately processed for MNC isolation and enrichment using density gradient centrifugation. The isolated MNC samples were CD3-depleted as previously described in Example 3 and cryopreserved in 20-25 mL Control In-Process Cryoprotectant Media, comprised of 10% DMSO, 6.25% w/v HSA, and 40% Plasmalyte-A, in cryobags at concentrations of 0.7×106 cells/mL to 1.4×106 cells/mL. During clinical manufacturing, and based on the results of the study described in Example 4, two normal donors were collected by apheresis and the MNC preparations were CD3-depleted following large-scale clinical procedures on the Miltenyi CliniMACS® device. The CD3-depleted MNCs were cryopreserved in 50 to 100 cryovial aliquots (1.8 mL each) in Test In-Process Cryoprotectant Media, comprised of 10% DMSO and 40% FBS in 50% RPMI Complete Media with GlutaMAX at a concentration of 83×106 cells/mL. All sample fractions were stored below −150° C., in a LN₂ vapor phase freezer, for a minimum of 24 hours. A freezer program was used and successful controlled freeze was verified by a freeze curve.

Samples were thawed as previously described in Example 4. Manual cell counts were performed to assess post-thaw percent cell viability and percent cell recovery. The results showed that CD3-depleted MNCs cryopreserved in the Test In-Process Cryoprotectant Media (10% DMSO and 40% FBS in 50% RPMI Complete Media with GlutaMAX) resulted in improved percent cell recovery with a mean 75% (±8.9%; median 74%), compared to CD3-depleted MNCs cryopreserved in Control In-Process Cryoprotectant Media (10% DMSO, 6.25% (w/v) HSA, and 40% Plasmalyte-A) a mean of 42% (±5.4%; median 42%) (Table 9). Similar results were observed for percent cell viability (data not shown).

TABLE 9
Comparison of In-Process Cryoprotectant Media on CD3-Depleted MNCs
After Cryopreservation Cryobags, Thawing, And Washing
Cell Recovery (%)

| | Control In-Process | Test In-Process |
|---|---|---|
| | Cryoprotectant | Cryoprotectant |
| Media (volume) | Media (20-25 mL) | Media (1.8 mL) |
| CD3-Depleted | 0.7 × 10⁶ cells/mL − | 83 × 10⁶ cells/mL |
| MNCs | 1.4 × 10⁶ cells/mL | |
| Median | 42% | 74% |
| Mean | 42% | 75% |
| Std Dev | 5.4% | 8.9% |

In conclusion, the results obtained during pre-clinical testing showing improved viable cell recovery in the Test In-Process Cryoprotectant Media (10% DMSO and 40% FBS in 50% RPMI Complete Media with GlutaMAX) have continued during clinical manufacturing.

### Example 6

A commercial cryoprotectant media and commercial thaw media were compared to a control media and evaluated for use in cryopreserving and thawing both T cell-depleted MNCs and NK cells during (in-process) and after NK cell expansion.

The following cryopreservation media were evaluated for MNCs: (1) Amsbio's STEM-CELLBANKER™, which reportedly comprises 5-10% DMSO and ≤10% inorganic salts and (2) In-Process Cryoprotectant Media comprising, 10% DMSO and 40% FBS in 50% RPMI Complete Media. The following thaw media were evaluated for MNCs: (1) BioLife Solutions Cell Thawing Media®, which reportedly comprises 10% dextran 40 in 5% dextrose and (2) In-Process Thaw Media comprising, RFG-NK Expansion Media and 50 U/mL DNase with 400 mg/mL MgCl₂ (RFG-NK Expansion Media is comprised of RPMI Complete Media with GlutaMAX and 10% FBS).

The following cryopreservation media were evaluated for NK cells during (in-process) and after NK cell expansion: (1) STEM-CELLBANKER™ and (2) NK Cell Cryopreservation Media comprising, 10% DMSO, 6.25% (w/v) HSA, and 40% Plasmalyte-A. The following thaw media were evaluated for NK cells: (1) BioLife Solutions Cell Thawing Media®, (2) In-Process Thaw Media comprising, RFG-NK Expansion Media and 50 U/mL DNase with 400 mg/mL MgCl₂, and (3) Final NK Cell Thaw Media comprising, 5% (w/v) HSA in 0.9% preservative-free normal saline.

Fresh whole blood specimens were collected from three healthy donors and immediately processed for MNC isolation and enrichment using density gradient centrifugation. The MNC samples were CD3-depleted as previously described in Example 3. Following CD3-depletion, each of the samples were split into several fractions prior to cryopreservation and stored in cryovials. Samples were diluted with either In-Process Cryoprotectant Media (10% DMSO and 40% FBS in 50% RPMI Complete Media) or STEM-CELLBANKER™, to an average concentration of 6.9×10⁶ cells/cryovials. The Control MNC samples (1.8 mL total volume/cryovial) were cryopreserved below −150° C., in a LN₂ vapor phase freezer using a freezer program (controlled rate freezer). Successful controlled freeze was verified by a freeze curve. The STEM-CELLBANKER™ MNC samples were cryopreserved according to the manufacturer's instructions (1 mL total volume/cryovial), first placed into a Stratacooler®, previously chilled to 4° C., and placed into −80° C. storage overnight before being relocated to LN₂ storage (controlled rate freezer not required). MNC samples were cryo-stored for a minimum of 3 days.

The samples of MNCs were thawed in a 37° C. water bath and diluted 1:5 with either BioLife Solutions Cell Thawing Media® or Thaw Media (RFG-NK Expansion Media and 50 U/mL DNase with 400 mg/mL MgCl₂). MNCs were washed by centrifuging at 300×g for 10 minutes at room temperature with a 50% brake and resuspended in 10 mL of the same Thaw Media. Manual cell counts were performed after thaw and wash.

RFG-NK Expansion Media comprising, 50 mL high quality (HQ) FBS, 5 mL GlutaMAX in 500 mL sterile RPMI 1640 Complete Media (with Phenol Red, and without L-glutamine) and IFCs were warmed to 37° C. 10 mL of RFG-NK Expansion Media was added to G-Rex10M cell culture devices, followed by 2×10⁴ U/mL IL-2 Working Solution (final concentration of 100 U/mL). The CD3-depleted MNC samples were seeded at 4×10⁶ cells. IFCs were washed in RFG-NK Expansion Media and added at a ratio of 2:1 (IFC to MNC). Last, additional RFG-NK Expansion Media was added to total media capacity. Incubator was maintained at 37° C., 5% CO₂. Media was exchanged and IL-2 was supplemented every two to three days during Stimulation 1 (days 0-7). At day 7, the expanding cells were harvested and split into two fractions. The fractions were washed in RFG-NK Expansion Media by centrifugation at 900×g for 10 minutes at room temperature.

One fraction was further split and cryopreserved, in either STEM-CELLBANKER™ or NK Cell Cryopreservation Media (10% DMSO, 50% (v/v) HSA, and 40% Plasmalyte-A), as previously described. The other fraction was re-seeded into new G-Rex® devices at a concentration of 2.10×10⁷ cells/G-Rex® device. IL-2 and RFG-NK Expansion Media were added as previously described. IFCs were added at a ratio of 1:1 (IFC to Stim1 cells). Cell culture was incubator maintained at 37° C., 5% CO₂. During Stimulation 2, cells were split based on number of cells and theoretical NK yield (calculated by multiplying actual yields times fraction factors/split ratios). If splitting is not performed, at least half of the cell culture device's media is exchanged and IL-2 is added, every 1 to 3 days. At 14 days, the NK cells were harvested, washed in NK Cell Harvest Media (0.5% (w/v) HSA in Plasmalyte A) or (0.9% preservative-free normal saline), and cryopreserved using the same procedure described for day 7. Day 14 NK samples were cryo-stored for a minimum of 24 hours. Manual cell counts were performed prior to cryopreservation on day 7 and day 14.

The samples of NK cells, from day 7 (Stim1 cells) and day 14, were thawed in a 37° C. water bath and diluted 1:5 with either (1) BioLife Solutions Cell Thawing Media® or (2) In-Process Thaw Media (RFG-NK Expansion Media and 50 U/mL DNase with 400 mg/mL MgCl₂) for NK cells expanded to day 7 (Stim1 cells) or (3) Final NK Cell Thaw Media (5% (w/v) HSA in 0.9% preservative-free normal saline) for NK cells expanded to day 14. Cells were washed by centrifuging at 300×g for 10 minutes at room temperature with a 50% brake and resuspended in 10 mL of the same Thaw Media. Manual cell counts were performed after thaw and wash.

TABLE 10
Comparison of Cryoprotectant Media and Thaw Media on CD3-
Depletec MNCs After Cryopreservation, Thawing, And Washing

| | Cryoprotectant Media | | | |
|---|---|---|---|---|
| | | In-Process | STEM-CELLBANKER ™ or In- | |
| | STEM- | Cryopreservation | Process Cryopreservation Media | |
| | CELLBANKER ™ | Media | | In-Process |
| Thaw Media | In-Process Thaw Media | | BioLife ® | Thaw Media |
| Post-Thaw Viability % | 93.5% (±6.0) | 97.8%(±0.8) | 80.22% (±9.6) | 95.0%(±4.7) |
| (Mean ± Std Dev) | | | | |
| Post-Thaw % Recovery | 70.2% (±0.9) | 73.7%(±6.6) | 11.6% (±7.8) | 58.4%(±29.3) |
| (Mean ± Std Dev) | | | | |

There was no statistical difference between the percent viability and percent recovery of CD3-depleted MNCs cryopreserved in STEM-CELLBANKER™ or the In-Process Cryoprotectant Media (10% DMSO and 40% FBS in 50% RPMI Complete Media) (Table 10), following thawing and washing in In-Process Thaw Media (RFG-NK Expansion Media and 50 U/mL DNase with 400 mg/mL MgCl₂).

Since there was no observed impact on post-thaw percent viability or percent recovery of CD3-depleted MNCs when cryopreserved in STEM-CELLBANKER™ or the Cryoprotectant Media, the groups were combined for analysis of thaw data. Cryopreserved CD3-depleted MNCs thawed and washed in BioLife Solutions Cell Thawing Media® or In-Process Thaw Media (RFG-NK Expansion Media and 50 U/mL DNase with 400 mg/mL MgCl₂) were evaluated for post-thaw percent viability or percent recovery. Post-thaw percent viability for BioLife®, mean 80.2% (±9.6) was significantly lower (p<0.01; unpaired t-test) than In-Process Thaw Media, mean 95.04% (±4.73) (Table 10). Post-thaw percent recovery for BioLife °, mean 11.6% (±7.8) was much lower (p=0.03; paired t-test) than In-Process Thaw Media, mean 58.4% (±29.3). Results indicate a significant decrease in performance by the BioLife Solutions Cell Thawing Media®.

TABLE 11
Comparison of Cryoprotectant Media and Thaw Media on Expanded
NK Cells After Cryopreservation, Thawing, And Washing

| | Cryoprotectant Media | | | |
|---|---|---|---|---|
| | NK Cells Harvested on Expansion Day 7 (Stim1 cells) | | | |
| | | In-Process | STEM-CELLBANKER ™ or In- | |
| | STEM- | Cryopreservation | Process Cryopreservation Media | |
| | CELLBANKER ™ | Media | | In-Process |
| Thaw Media | In-Process Thaw Media | | BioLife ® | Thaw Media |
| Post-Thaw Viability % | 56.9% (±24.6) | 60.6%(±16.7) | 26.2% (±18.7) | 58.74% (±19.6) |
| (Mean ± Std Dev) | | | | |
| Post-Thaw % Recovery | 54.5% (±20.7) | 72.9%(±15.6) | 7.8% (±4.2) | 62.8% (±20.1) |
| (Mean ± Std Dev) | | | | |
| | Cryoprotectant Media | | | |
| | NK Cells Harvested on Expansion Day 14 | | | |
| | | In-Process | STEM-CELLBANKER ™ or In- | |
| | STEM- | Cryopreservation | Process Cryopreservation Media | |
| | CELLBANKER ™ | Media | | Final NK Cell |
| Thaw Media | Final NK Cell Thaw Media | | BioLife ® | Thaw Media |
| Post-Thaw Viability % | 87.9% (±5.2) | 86.3% (±6.3) | 57.8% (±19.2) | 87.1% (±5.4) |
| (Mean ± Std Dev) | | | | |
| Post-Thaw % Recovery | 94.9% (±16.1) | 89.7% (±22.3) | 7.4% (±3.4) | 92.3% (±18.2) |
| (Mean ± Std Dev) | | | | |

There was no statistical difference in the average percent viability and percent recovery of NK cells harvested at day 7 (Stim1 cells), cryopreserved with STEM-CELLBANKER™ or In-Process Cryoprotectant Media (10% DMSO and 40% FBS in 50% RPMI Complete Media); after thawing and washing in In-Process Thaw Media (RFG-NK Expansion Media and 50 U/mL DNase with 400 mg/mL MgCl₂) (Table 10). Similarly, there was no statistical difference in the average percent viability and percent recovery of Expanded NK cells harvested at day 14, cryopreserved with STEM-CELLBANKER™ or NK Cell Cryopreservation Media (10% DMSO, 6.25% w/v HSA, and 40% Plasmalyte-A); after thawing and washing in Final NK Cell Thaw Media (5% (w/v) HSA in 0.9% saline). While there does not appear to be a significant improvement in using STEM-CELLBANKER™ over the Control Cryopreservation Media, the commercial media could potentially improve the logistics of aliquoting cells into their respective containers to be placed into cryopreservation by reducing the amount of processing time and exposure to DMSO.

Similar to the results for the CD3-depleted MNCs, as there was no observed impact on post-thaw percent viability or percent recovery of NK cells expanded to day 7 (Stim1 cells) or to day 14 when cryopreserved in STEM-CELLBANKER™ or the Control Cryoprotectant Media, the groups were combined for analysis of thaw data (Table 11). Thawing and washing the cryopreserved cells in BioLife Solutions Cell Thawing Media® revealed significantly lower percent viability and percent recovery than either the In-Process or Final NK Cell Thaw Media (p<0.01 for all groups; paired t-test) (Table 11). Thus, consistent with the MNC thaw data, results for NK cells expanded to day 7 (Stim1 cells) or day 14, indicate a significant decrease in performance by the BioLife Solutions Cell Thawing Media® compared to Thaw Media.

### Example 7

The following experiment was performed to verify that altering the IL-2 stimulation and counting from days 1 and 4 to days 3 and 5, during NK expansion, does not affect the 14 day NK cell yield and/or the immunophenotypes. In addition, stability at room temperature was assessed for fresh, Expanded NK cells and Expanded NK cells post-thaw. Fresh whole blood specimens were collected from healthy donors. The specimens were immediately processed for MNC isolation and enrichment using density gradient centrifugation. The isolated MNC samples were CD3-depleted as previously described in Example 3.

RFG-NK Expansion Media and IFCs were warmed to 37° C. in water bath. RFG-NK Expansion Media was added to G-Rex100 devices, followed by IL-2 Working Solution (100U/mL final), and CD3-depleted MNCs. IFCs were added at an IFC to MNC ratio of 2:1. Last, additional RFG-NK Expansion Media was added to reach total media capacity. Cell culture incubator maintained at 37° C., 5% CO₂. Cultures were stimulated with IL-2 on days 1 and 4 or days 3 and 5. Manual viable cell counts and immunophenotypic analysis was performed on day 0 and day 7.

On day 7, cells generated from Stimulation 1 (Stim1 cells) were harvested and seeded in new G-Rex100 devices. IL-2 and RFG-NK Expansion Media were added as previously described. IFCs were added at a ratio of 1:1 (IFC to Stim1 cells). Incubator maintained at 37° C., 5% CO₂. During Stimulation 2, cells were split and stimulated with IL-2 twice. Manual viable cell counts and immunophenotypic analysis was performed on day 13 or 14.

Results reveal that the alteration of IL-2 stimulation and counting schedule from days 1 and 4 to days 3 and 5 during NK expansion, does not affect the 14 day NK cell yield and/or the immunophenotypes. Specifically, the NK cells that were stimulated with IL-2 on days 1 and 4 showed a 14 day fold NK cell expansion of 3,186, and at day 13, percent T/NK T cells of 0.19%, percent NK cells of 95.1%, and day 13 FCs of 0.001%. The NK cells that were stimulated with IL-2 on days 3 and 5 showed a 14 day fold NK cell expansion of 2,4486, and at day 13, percent T/NK T cells of 0.26%, percent NK cells of 96.0%, and day 13 FCs of 0.002% (Table 12). No differences were seen, suggesting alterations in the IL-2 stimulation and counting schedule during Stimulation 1 does not affect the NK cell expansion process.

TABLE 12
Alternative Feed and Split Parameters During NK Cell Expansion

| | | |
|---|---|---|
| Stim1 count/IL-2 | day 1, day 4 | day 2, day 3 |
| Stim1 Fold NK | 67 | 30 |
| Expansion | | |
| Stim2 IL-2 ± Split | day 8, day 11 | day 10, day 12 |

| | |
|---|---|
| Stim2 Split | Maintain < 800/G-Rex100 |
| Concentration | |

| | | |
|---|---|---|
| Split(s) performed | 1:3 | 1:2 & 1:2 |
| Stim2 Fold NK | 47 | 82 |
| Expansion | | |
| 14 Day Fold NK | 3,186 | 2,448 |
| Expansion | | |
| Day 13 T/NK T cells | 0.19% | 0.26% |
| Day 13 NK cells | 95.1% | 96.0% |
| Day 13 FCs | 0.001% | 0.002% |

After harvest, a fraction of the Expanded NK cells were cryopreserved for a minimum of 24 hours, as previously described, then thawed (no wash) and tested for post-thaw stability at room temperature from 0.5 hour to 4 hours. The other fraction of fresh Expanded NK cells were tested for stability at room temperature at 24, 48, and 72 hours. Cell viability and immunophenotypic analysis were performed.

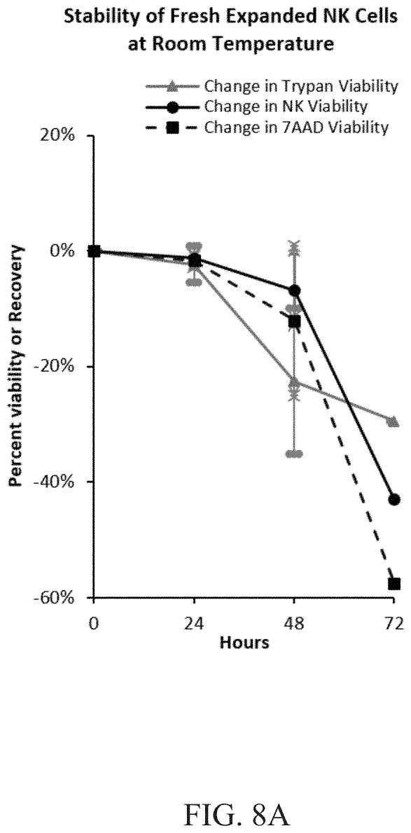

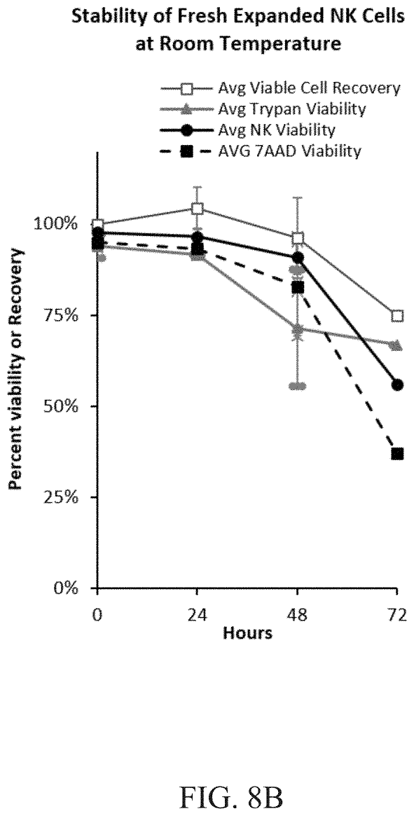

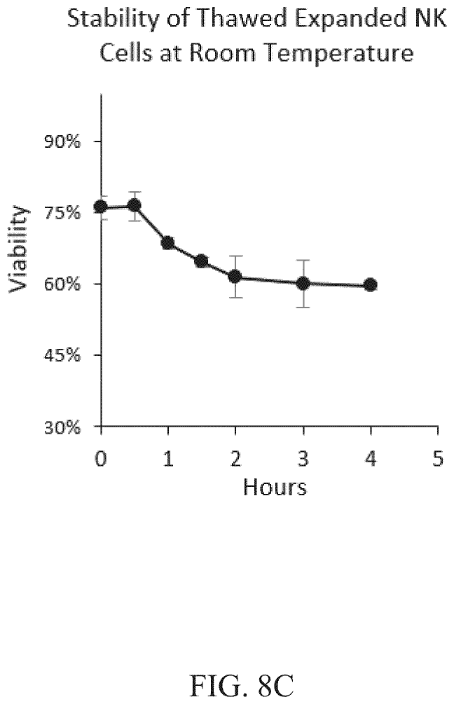

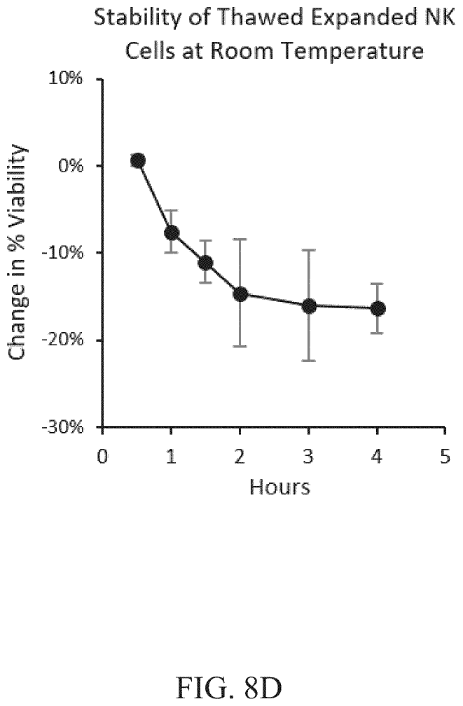

Stability results for fresh Expanded NK cells at room temperature revealed that while percent cell viability dropped an average of 23% after 48 hours (FIG. 8A), both WBC and NK percent viability by flow were more stable, dropping an average of 12% and 7% respectively after 48 hours (FIG. 8B). Stability results for fresh Expanded NK cells at room temperature revealed that percent viability decreased approximately 25% immediately after thaw, but was stable for the first 30 minutes, after which time viability decreased another 10% (FIG. 8C). Viable cell recovery was more variable and decreased steadily for the first 1.5 hours before leveling out at approximately 60% recovery (FIG. 8D). Microbial cultures of samples taken 4 hours post-thaw were negative.

### Example 8

Fresh whole blood specimens were collected from healthy donors and immediately processed for MNC isolation and enrichment using density gradient centrifugation. The same day, MNC samples underwent CD3-depletion using the Miltenyi Biotec SuperMACS® Magnet System with XS column. Manual viable cell counts and immunophenotypic analysis was performed before and after CD3-depletion. After CD3-depletion the percentage of T cells and NK T cells (CD3+ cells) per total WBCs (CD45+ cells) decreased from 12.53% to 0.58%. The percentage of NK cells (CD3-CD56+ cells) per total WBCs (CD45+ cells) increased from 4.36% to 74.08%. A total of 18.6×10⁶ WBCs (13.8×10⁶ NK cells) were isolated from 90 mL fresh blood, with an NK cell recovery of 69%.

CD3-depleted MNCs were seeded into four G-Rex cell culture devices (sample IDs: 10.a, 10.b, 10.c, and 10.d) and co-cultured with IFCs (Table 13). The following reagents were used: RFG-NK Expansion Media, 50 mL high quality (HQ) FBS, 5 mL GlutaMAX in 500 mL sterile RPMI 1640 Complete Media (with Phenol Red, and without L-glutamine); and IL-2 Working Solution comprising about 2×10⁴ U IL-2 per mL of RFG-NK Expansion Media.

RFG-NK Expansion Media and IFCs were warmed to 37° C. in water bath. 10 mL of RFG-NK Expansion Media was added to each G-Rex® device, followed by 2×10⁴ U/mL IL-2 Working Solution (0.2 mL for G-Rex10; 0.5 mL for G-Rex10M), CD3-depleted MNCs (4×10⁶ cells/G-Rex® device). IFCs were washed in RFG-NK Expansion Media and added to the G-Rex® devices (IFC to MNC ratio of 2:1). Last, additional RFG-NK Expansion Media was added to G-Rex® devices to reach total media capacity. Cell culture incubator maintained at 37° C., 5% CO₂. Culture conditions during Stimulation 1 (days 0-7) are outlined in Table 13. Manual viable cell counts and immunophenotypic analysis was performed on day 7.

TABLE 13
NK Cell Expansion Stimulation1 Comparison of Different Handling Conditions

| | | | Cell | Day 7 NK | Day 7 NK | Day 7 T/NK |
|---|---|---|---|---|---|---|
| Sample ID | Day 3 | Day 5 | Viability | Cell Yield | Cells % | T Cells % |
| 10.b | Mixed, | Mixed, | 84.3% | 1.5 × 10⁸ | 89.92% | 0.76% |
| | Counted, IL-2 | Counted, IL-2 | | | | |
| 10.c, 10.d (avg) | IL-2 | IL-2 | 81.2% | 1.5 × 10⁸ | 91.77% | 0.84% |

Comparing two different handling conditions on days 3 and 5: (1) mixing, counting and adding IL-2 vs. (2) only adding IL-2, revealed no significant difference in percent cell viability, NK Cell yield, percent NK cells, or percent T/NK T cells (Table 13). Only samples cultured in the same G-Rex10M device are shown in Table 13. Samples 10.c and 10.d were averaged. These results indicate that mixing the cells during Stim1 generally does not affect NK expansion. Sample 10.a, cultured in the short G-Rex10 device, had reduced percent cell viability (78.6%), NK Cell yield (4.26×10⁷ NK cells), and percent NK cells (86.5%), and increased percent T/NK T cells (2.03%) compared to samples cultured in the tall, G-Rex10M.

Cells generated from Stimulation 1 (Stim1 cells) were harvested and seeded in new G-Rex® devices at a concentration of 2.10×10⁷ cells/G-Rex® device. IL-2 and RFG-NK Expansion Media were added as previously described. A duplicate sample was added to each of the four groups. IFCs were added at a ratio of 1:1 (IFC to Stim1 cells). Incubator maintained at 37° C., 5% CO₂. During Stimulation 2, cells were split based on number of cells and theoretical NK yield (calculated by multiplying actual yields times fraction factors/split ratios). Culture conditions during Stimulation 2 (days 7-14), including split day are outlined in Table 14. Manual viable cell counts and immunophenotypic analysis was performed again on day 14.

TABLE 14
NK Cell Expansion: Stimulation2 Conditions and Comparison of
Short vs. Tall G-Rex ® Cell Culture Devices

| | | | Device Height | | Mean 14 Day | | |
|---|---|---|---|---|---|---|---|
| | | G-Rex ® Device | (Total Media | | NK Cell Fold | Mean Day 14 | Day 14 T/NK |
| Sample ID (n) | | (Surface Area) | Capacity) | | Expansion | NK Cells % | T Cells % |
| 10.a | (n = 2) | G-Rex10 | Short | (40 mL) | 525 | 97.73% | 0.53% |
| | | (10 cm²) | | | | | |
| 10.b-10.d | (n = 6) | G-Rex10M | Tall | (100 mL) | 2617 | 96.65% | 0.66% |
| | | (10 cm²) | | | | | |

Analysis of NK cells on day 14 revealed that the tall (100 mL), G-Rex10M devices resulted in a 5× greater yield of total NK cells compared to the short (40 mL), G-Rex10 devices (Table 14). No differences in percent NK cells or percent T/NK T cells was observed. The data was also analyzed for any affects due to splitting once or twice during Stim2. Among samples cultured in the tall, G-Rex10M devices no statistically significant differences were observed between splitting once vs. twice for 14 day NK fold expansion, percent NK cells, or percent T/NK T cells (data not shown).

### Example 9

The following study was performed retrospectively to compare two different sized G-Rex® cell culture devices used during pre-clinical validations, thus adding to the data shown in Example 7.

The following experiment was performed to compare short, G-Rex10 devices vs. tall, G-Rex10M devices during NK expansion. Fresh whole blood specimens were collected from healthy donors. The specimens were immediately processed for MNC isolation and enrichment using density gradient centrifugation. The isolated MNC samples were CD3-depleted as previously described in Example 3.

RFG-NK Expansion Media and IFCs were warmed to 37° C. in water bath. RFG-NK Expansion Media was added to each G-Rex® device, followed by IL-2 Working Solution (100U/mL final), and CD3-depleted MNCs. IFCs were washed in RFG-NK Expansion Media and added to the G-Rex® devices (IFC to MNC ratio of 2:1). Last, additional RFG-NK Expansion Media was added to G-Rex® devices to reach total media capacity. Cell culture incubator maintained at 37° C., 5% CO₂. Cultures were stimulated with IL-2, and short G-Rex devices were fed (media exchange), every 2-3 days. Manual viable cell counts and immunophenotypic analysis was performed on day 0 and day 7.

On day 7, cells generated from Stimulation 1 (Stim1 cells) were harvested and seeded in new G-Rex® devices. IL-2 and RFG-NK Expansion Media were added as previously described. IFCs were added at a ratio of 1:1 (IFC to Stim1 cells). Incubator maintained at 37° C., 5% CO₂. During Stimulation 2, cells were split, stimulated with IL-2, and short G-Rex devices were fed (media exchange), every 1-3 days. Manual viable cell counts and immunophenotypic analysis was performed on day 13 or 14. Data were further analyzed to determine the yield of NK cells per mL of whole blood collected and the number of doses at 1×10⁸ NK cells per kg when normalized for a 50 kg recipient and 150 mL blood draw.

Results show that when NK cell expansion is performed in tall G-Rexx devices (e.g., G-Rex10M), a greater fold-expansion of NK cells after Stim2 is achieved from day 7 to day 14, compared to short G-Rex® devices (e.g., G-Rex10) with an average of 58.3-fold expansion compared to 33.8-fold (Table 15). Although there was no difference in the Stim1 Fold NK Cell Expansion observed in this larger data set as was seen in parallel testing, the results from Stim2 (day 7 to day 14) more than overcome this, with overall 14 day fold expansion averaging 2663-fold in tall G-Rex® devices compared to 891-fold in short devices. Moreover, an average of 10.1 normalized doses of 1×10⁸ NK cells per kg was obtained from tall G-Rex® devices compared to just 2.0 from short devices.

TABLE 15
Short vs. Tall G-Rex ® Cell Culture Devices

| | | Stim1 Fold | Stim2 Fold | 14 Day NK | NK Cell | |
|---|---|---|---|---|---|---|
| | | NK Cell | NK Cell | Cell Fold | Yield/mL | Normalized NK |
| G-Rex ® Device | Results | Expansion | Expansion | Expansion | Whole Blood | Cells/Dose* |
| Short, G-Rex10 | Mean | 54.3 | 33.8 | 891 | 0.7 × 10⁸ | 2.0 |
| (10 cm² surface area; | Median | 57 | 27.5 | 854 | 0.6 × 10⁸ | 1.8 |
| 40 mL total vol.) | | | | | | |
| Tall, G-Rex10M | Mean | 47.2 | 58.3 | 2663 | 3.4 × 10⁸ | 10.1 |
| (10 cm² surface area; | Median | 57 | 79.5 | 2611 | 3.4 × 10⁸ | 10.2 |
| 100 mL total vol.) | | | | | | |

The invention is claimed as follows:

1. A method of expanding natural killer (NK) cells, the method comprising:
growing a culture of T cell-depleted mononuclear cells, the culture comprising a cell nutrient media and at least one activating agent;
splitting the culture of T cell-depleted mononuclear cells at least once after seven days;
replacing the cell nutrient media at least once after seven days; and
harvesting Final NK cells after about 14 days, the Final NK cells comprising less than about 5% of CD3+ cells/white blood cells (WBCs) and more than about 90% of CD3-CD56+ cells/WBCs.

2. The method of claim 1, wherein the Final NK cells comprises an average of 2,800 times more NK cells compared to the T cell-depleted mononuclear cells.

3. The method of claim 1, wherein the at least one activating agent is provided to the culture of T cell-depleted mononuclear cells by a delivery vehicle.

4. The method of claim 1, wherein the Final NK cells comprise less than about 1% of CD3+ cells/WBCs and more than about 95% of CD3-CD56+ cells/WBCs.

5. A therapeutic composition comprising:
cryopreserved Final NK cells including less than about 5% of CD3+ cells/WBCs and more than about 90% of CD3-CD56+ cells/WBCs, wherein HLA type of the cryopreserved Final NK cells is known; and
wherein the composition of Final NK cells comprises more than one concentration of cryopreserved Final NK cells.

6. The composition of claim 5, wherein the Final NK cells have a concentration selected from 1×10⁷ cells/mL, 3×10⁷ cells/mL, or 1×10⁸ cells/mL.

7. The composition of claim 5, wherein the composition comprises less than about 1% of CD3+ cells/WBCs and more than about 95% of CD3-CD56+ cells/WBCs.

8. A method of storing a therapeutic composition, the method comprising:
cryopreserving Final NK cells in a cryopreservation media, the Final NK cells comprising less than about 5% of CD3+ cells/WBCs and more than about 90% of CD3-CD56+ cells/WBCs, wherein Final NK are a known HLA-type; and
wherein the therapeutic composition comprises more than one concentration of Final NK cells.

9. The method of claim 8, wherein the cryopreservation media comprises, 10% DMSO (v/v) and 12.5% (w/v) HSA in Plasmalyte-A.

10. A method of treating a patient, the method comprising:
determining the patient's HLA-specific antibody profile,
excluding a Final NK cell composition that may be cross-reactive with the patient's HLA-specific antibody profile;
measuring the patient's weight or body surface area;
calculating the total number of NK cells to be administered to the patient based on the patient's weight or body surface area;
selecting at least one Final NK cell composition according to the total number of NK cells to be administered to the patient; and
administering the Final NK cell composition to the patient.

11. The method of claim 10, wherein the patient has a cancer or a tumor.

12. The method of claim 10, wherein the patient has an infectious disease.

13. A system for determining the Final NK Cell Composition ID to be administered to a patient, the system comprising:
a memory device storing patient data, clinical objective and acceptance data, donor data, and Final NK Cell Composition ID data;
a clinical decision algorithm configured to determine the Final NK Cell Composition IDs to be administered to the patient;
a processor communicatively coupled to the memory device, the processor in conjunction with the clinical decision algorithm configured to:
determine incompatibilities between the patient and the Final NK Cell Composition ID, calculate a minimum and maximum NK cell dose range, and determine the Final NK Cell Composition IDs to be administered to the patient.

14. The system of claim 13, wherein the patient data includes at least one of a weight in kg, body surface area in m², a dose of NK cells/kg, or a dose of NK cells/m².

15. The system of claim 13, wherein the clinical objective and acceptance data includes at least one of a maximum CD3+ cell dose in cells/kg or a minimum and maximum NK cell dose range in cells/kg.

16. The system of claim 13, wherein the donor data includes at least one of a specimen collection date or an HLA-type.

17. The system of claim 13, wherein the Final NK Cell Composition ID data includes at least one of a total number of CD3-CD56+ cells/mL, a percentage of CD3+ cells/WBCs, or a total storage volume in mL.
